(19) United States
(12) Patent Application Publication
(10) Pub. No.: US 2020/0357486 A1
(43) Pub. Date: Nov. 12, 2020

(54) OPTIMIZATION OF ORGANISMS FOR PERFORMANCE IN LARGER SCALE CONDITIONS BASED ON PERFORMANCE IN SMALLER SCALE CONDITIONS

(71) Applicant: Zymergen Inc., Emeryville, CA (US)

(72) Inventors: Stefan De Kok, Berkeley, CA (US); Peter Enyeart, Emeryville, CA (US); Richard Hansen, San Carlos, CA (US); Trent Hauck, Seattle, WA (US); Zachariah Serber, Kenwood, CA (US); Amelia Taylor, Bend, OR (US); Thomas Treynor, Berkeley, CA (US); Kristina Tyner, Richmond, CA (US); Sarah Lieder, Oakland, CA (US)

(73) Assignee: Zymergen Inc., Emeryville, CA (US)

(21) Appl. No.: 16/762,022

(22) PCT Filed: Nov. 9, 2018
(86) PCT No.: PCT/US18/60120

§ 371 Date: May 6, 2020

Related U.S. Application Data
(60) Provisional application No. 62/583,961, filed on Nov. 9, 2017.

(51) Int. Cl. G16B 40/20; G06N 20/00

(52) U.S. Cl. CPC G16B 40/20; G06N 20/00

(57) ABSTRACT

Systems, methods and computer-readable media storing executable instructions are provided for improving performance of an organism with respect to a phenotype of interest at a second scale based upon measurements at a first scale. First scale performance data based at least in part upon observed first performance of first organisms at a first scale and second scale performance data based at least in part upon observed second performance of second organisms at a second scale larger than the first scale are accessed. A prediction function based at least in part upon the relationship of the second scale performance data to the first scale performance data is generated. The prediction function may be applied to performance data observed for test organisms with respect to the phenotype of interest at the first scale to generate second scale predicted performance data for the test organisms at the second scale.

### CROSS-REFERENCE TO RELATED APPLICATIONS

This application claims the benefit of priority to U.S. provisional application No. 62/583,961, filed Nov. 9, 2017, which is hereby incorporated by reference in its entirety.

### BACKGROUND

### Field of the Disclosure

The disclosure relates generally to the fields of metabolic and genomic engineering, and more particularly to the field of metabolic optimization of organisms for production of chemical targets in large-scale environments.

### Description of Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

The best approach for optimizing the performance of an incompletely understood system, such as a living cell, is often to test many as many different modifications as possible and empirically determine which perform best. Since testing modifications at a scale relevant to industrial production is typically expensive and time-consuming, the throughput for testing modifications at scale is very low. Therefore, small-scale, high-throughput screening approaches are used to quickly identify the best candidates for performance from among large numbers of modifications. For this approach to be successful, however, there must be a reliable means of predicting larger-scale performance from smaller-scale performance. As examples, the scales range from small plates with many wells (e.g., 200-μL per well), to larger plates with fewer wells, to bench-scale tanks (e.g., 5 or more liters), to industrial-sized tanks (e.g., 100-500,000 liters).

A technical field where such approaches have been widely applied is in the pharmaceutical industry, for purposes of identifying new and useful drugs. Thousands of candidate molecules may be first screened in vitro for activity in an assay that is expected to be a predictive proxy for in vivo activity. Statistical approaches are applied to determine the best performers (see, for example, Malo et al. “Statistical practice in high-throughput screening data analysis.” Nat Biotechnol 24:167-175 (2006)), which are then used in more expensive, larger scale experiments, which may include in vivo testing in mice and humans.

However, these approaches are geared toward binary judgments (e.g., effective or not effective) as opposed to ranking performance for future decisions regarding a lower-throughput experiment. Further, these approaches assume that the vast majority of tested samples will have the same value and will not be of interest. In the field of metabolic engineering, where the genetic pathways of a cell are optimized to produce a specific product of interest at scale, these assumptions do not hold. In particular, when iteratively adding improvements to multiple strain lineages, the measured values may vary widely, and there may be far more samples that seem to be improvements than can be reasonably screened at a large scale at lower throughput and, as such, clear ranking of performance is required. In other words, it is not enough to determine which samples are better; it is important to know which samples are best, and preferably by how much, at the next level of scale.

### SUMMARY OF THE DISCLOSURE

In conventional predictive modeling, statistical outliers are typically removed from the training data set to reduce predictive error of the model. However, the inventors have recognized that, in the field of genomic engineering, discarding such outliers may not be necessary to achieve the optimal model for predicting performance in larger scale conditions from smaller scale conditions. Instead, further features may be added to the model to mitigate the need to remove outliers.

The present disclosure provides a robust method for reliably predicting the values of key performance indicators (e.g., yield, productivity, titer) in larger-scale, low-throughput conditions based on smaller-scale, high-throughput measurements, especially in the technical field of metabolic optimization of organisms for mass-production of chemical targets. Embodiments of the disclosure may employ an optimized statistical model for the prediction. Further, the present disclosure provides a transfer function development tool that produces the model in a reproducible way, records decisions, and provides a fast and easy mechanism for getting and working with the predicted values.

In the context of this disclosure, a transfer function is a statistical model for predicting performance in one context based on performance in another, where the primary goal is to predict the performance of samples at a larger-scale from their performance at smaller-scale. In embodiments, the transfer function employs a one-factor linear regression that considers the small-scale and large-scale values, along with optimizations discovered by the inventors. In other embodiments, the transfer function may employ multiple regression.

To build these regression models, some embodiments of the disclosure use a model to summarize the performance of a strain in the high-throughput context (e.g., a plate model), and then use a separate model (e.g., a transfer function) to predict the performance of a strain across multiple runs in the lower-throughput context.

In embodiments, particularly those employing a linear model for the transfer function, removing some strains from consideration was found to improve the predictive power of the model, and this iterative process has been its own optimization. In embodiments, methods using the sample characteristics listed above provide a mechanism for iteratively identifying characteristics (such as genetic modifications present, lineage, etc.) whose inclusion as a factor in predicting high-throughput performance allows for even more improvement in the predictive power, while also allowing strains to be kept in the model that otherwise might be removed. Such techniques ease the processing load in computing the predicted performance.

Embodiments of the disclosure provide systems, methods, and computer-readable media storing executable instructions for improving performance of an organism with respect to a phenotype of interest at a second scale based upon measurements at a first scale. Embodiments of the disclosure (a) access first scale performance data representing observed first performance of one or more first organisms at a first scale and second scale performance data representing observed second performance of one or more second organisms at a second scale larger than the first scale; and (b) generate a prediction function based at least in part upon the relationship of the second scale performance data to the first scale performance data. According to embodiments of the disclosure, the prediction function is applied to performance data observed for one or more test organisms with respect to the phenotype of interest at the first scale to generate second scale predicted performance data for the one or more test organisms at the second scale. Embodiments of the disclosure further comprise manufacturing at least one of the one or more test organisms based at least in part upon the second scale predicted performance.

According to embodiments of the disclosure, the first scale is a plate scale and the second scale is a tank scale. The one or more second organisms may be a subset of the one or more first organisms. The phenotype may includes production of a compound. The organism may be a microbial strain.

According to embodiments of the disclosure, the first scale performance data for the one or more first organisms is generated using a first scale statistical model. The first scale statistical model may represent organism features at the first scale. The organism features may comprise process conditions, media conditions, or genetic factors. The organism features may relate to organism location. According to embodiments of the disclosure, the prediction function is based at least in part upon a weighted sum of one or more first scale performance variables, wherein at least one of the first scale performance variables is based on a combination of two or more measurements of organism performance. (It is understood that the “sum of one or more” variables is just the variable itself when only one variable is being summed.) According to embodiments of the disclosure, the combination is based at least in part upon a ratio of product concentration to sugar consumption.

According to embodiments of the disclosure, generating the prediction function may comprise removing from consideration the first scale performance data and the second scale performance data for one or more outlier organisms. According to embodiments of the disclosure, generating the prediction function may comprise incorporating one or more factors (e.g., genetic factors) to reduce error (e.g., leverage metric) of the prediction function.

Embodiments of the disclosure may modify the prediction function by one or more factors from a set of factors; and exclude, from consideration in generating the prediction function, a first candidate outlier organism (i.e., exclude the observed performance data for the first candidate outlier organism) which, if included in generating the prediction function, would result in the modified prediction function having a leverage metric that fails to satisfy a leverage condition. According to embodiments of the disclosure, “leverage” may generally refer to the amount of influence that a strain has on the output of a predictive model (e.g., the predicted performance), including the effect on error in the predictive ability of the model. According to embodiments of the disclosure, if the leverage metric for the modified prediction function with respect to a first candidate outlier organism satisfies the leverage condition, such embodiments may use the modified prediction function as the prediction function.

According to embodiments of the disclosure, the first candidate outlier organism is an organism which, if excluded from consideration in generating the prediction function, leads to a greatest improvement in the leverage metric for the modified prediction function. Embodiments of the disclosure (a) identify as a second candidate outlier organism an organism which, if excluded from consideration in generating the prediction function with the first candidate outlier organism also excluded, leads to a greatest improvement in the leverage metric for the prediction function; (b) modify the prediction function by one or more factors from a set of factors to generate a second modified prediction function; and (c) exclude, from consideration in generating the prediction function, the second candidate outlier organism which, if included in generating the prediction function, would result in the second modified prediction function having a leverage metric that fails to satisfy a leverage condition.

According to embodiments of the disclosure, a first candidate outlier organism is represented in the first scale performance data and the second scale performance data, the one or more test organisms comprise the first candidate outlier organism, and the second scale predicted performance data represents predicted performance of the first candidate outlier organism at the second scale.

According to embodiments of the disclosure, modifying the prediction function comprises incorporating or removing the one or more factors respectively into or from the prediction function. According to embodiments of the disclosure, generating the prediction function comprises training a machine learning model using the first scale performance data and the second scale performance data. According to embodiments of the disclosure, generating the prediction function comprises applying machine learning in the process of modifying the prediction function by the one or more factors.

Embodiments of the disclosure compare performance error metrics for a plurality of prediction functions, and rank the prediction functions based at least upon the comparison.

According to embodiments of the disclosure the first scale performance data for the one or more first organisms represents the output of a first scale statistical model, and such embodiments compare predicted performance for the one or more first organisms at the second scale with the second scale performance data, and adjust parameters of the first scale statistical model based at least in part upon the comparison.

Embodiments of the disclosure provide an organism with improved performance of the phenotype of interest at the second scale, where the organism is identified using any of the method disclosed herein.

Embodiments of the disclosure provide a transfer function development tool that provides a user interface for user control of the development of a predictive model for an organism at a second scale based upon data observed at a first scale smaller than the second scale. According to embodiments, the tool also applies the prediction function to predict organism performance at the second scale.

Embodiments of the disclosure access a prediction function, wherein the prediction function is based at least in part upon the relationship of second scale performance data to first scale performance data, and may include optimizations such as outlier removal and incorporation of factors, such as genetic factors, as described herein. The first scale performance data represents observed first performance of one or more first organisms at a first scale, and the second scale performance data represents observed second performance of one or more second organisms at a second scale larger than the first scale. Such embodiments apply the prediction function to one or more test organisms at the first scale to generate second scale predicted performance data for the one or more test organisms at the second scale.

### BRIEF DESCRIPTION OF THE DRAWINGS

FIG. 1 illustrates a client-server computer system for implementing embodiments of the disclosure.

FIG. 2A illustrates a comparison of measured bioreactor (tank, larger scale) vs. plate (smaller scale) values for individual strains, according to embodiments of the disclosure.

FIG. 2B illustrates a comparison of actual tank yield values to linear predicted tank yield values for a bioreactor (tank) in an example according to embodiments of the disclosure.

FIG. 3 is a plot equivalent to that of FIG. 2B, except with Type 1 outlier strain N removed.

FIG. 4 is a plot equivalent to that of FIG. 2B, except with four Type 1 outliers and one Type 2 outlier removed.

FIG. 5 depicts the result of applying a correction to all the strains in FIG. 4 based on whether or not they have a certain genetic modification, according to embodiments of the disclosure.

FIG. 6 is a regression plot of the model shown in FIG. 5, according to embodiments of the disclosure.

FIG. 7 illustrates a productivity model without correction for genetic factors, according to embodiments of the disclosure.

FIG. 8 illustrates the productivity model of FIG. 7 after correction for a genetic factor, according to embodiments of the disclosure.

FIG. 9 illustrates improvement in the high-throughput productivity-model performance (x-axis) versus improvement in actual productivity in low-throughput bioreactors (e.g., tanks) (y-axis) for strains harboring the same promoter swap as in FIG. 8.

FIG. 10 illustrates a user interface of a transfer function development tool according to embodiments of the disclosure.

FIG. 11 illustrates the user interface, according to embodiments of the disclosure.

FIG. 12 illustrates a user interface displaying a plate-tank correlation transfer function, according to embodiments of the disclosure.

FIG. 13 illustrates the user interface presenting ten strains having the highest predicted performance based upon the transfer function with the outliers selected by the user having been removed from the model, according to embodiments of the disclosure.

FIG. 14 illustrates a graphical representation of the chosen transfer function after user-selected outliers have been removed from the model, according to embodiments of the disclosure.

FIG. 15 illustrates an interface enabling the user to submit quality scores for the removed strains to a database, according to embodiments of the disclosure.

FIG. 16 illustrates a cloud computing environment according to embodiments of the disclosure.

FIG. 17 illustrates an example of a computer system that may be used to execute program code to implement embodiments of the disclosure.

FIG. 18 is a graph of plate vs. tank values resulting from an experiment performed according to embodiments of the disclosure.

FIG. 19 is a graph of plate vs. tank values resulting from an experiment performed according to embodiments of the disclosure.

FIG. 20 is a graph of plate vs. tank values resulting from an experiment performed according to embodiments of the disclosure.

FIG. 21 is a graph of plate vs. tank values resulting from an experiment performed according to embodiments of the disclosure.

FIG. 22 is a graph of plate vs. tank values resulting from an experiment performed according to embodiments of the disclosure.

FIG. 23 is a graph of observed tank values vs. predicted tank values resulting from an experiment performed according to embodiments of the disclosure.

FIG. 24 is a graph of observed tank values vs. predicted tank values resulting from an experiment performed according to embodiments of the disclosure.

FIG. 25 is a graph plotting a first tank value vs. a second tank value resulting from an experiment performed according to embodiments of the disclosure.

FIG. 26 is a graph of observed tank values vs. predicted tank values resulting from an experiment performed according to embodiments of the disclosure.

FIG. 27 plots sugar (Cs), product (Cp) and biomass (Cx) concentrations that were estimated over time according to a prophetic example based on embodiments of the disclosure.

FIG. 28 is a graph of product concentration vs. fermenter product yield according to a prophetic example based on embodiments of the disclosure.

FIG. 29 is a graph of sugar concentration vs. fermenter product yield according to a prophetic example based on embodiments of the disclosure.

FIG. 30 is a graph of biomass concentration vs. fermenter product yield according to a prophetic example based on embodiments of the disclosure.

FIG. 31 is a graph of product yield in plates vs. fermenter product yield according to a prophetic example based on embodiments of the disclosure.

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

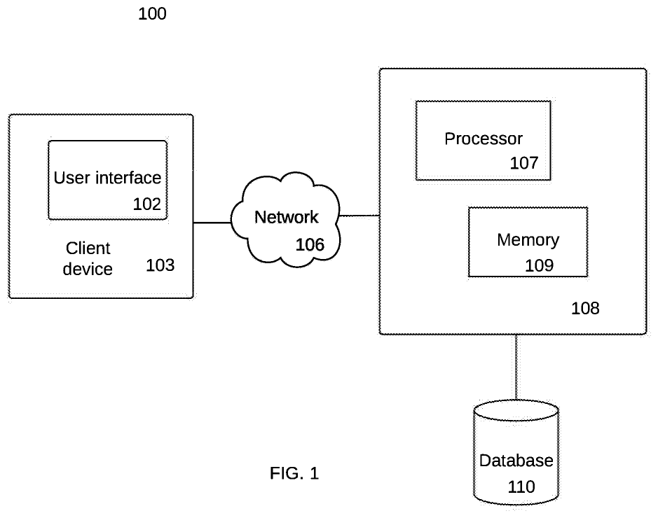

FIG. 1 illustrates a distributed system 100 of embodiments of the disclosure. A user interface 102 includes a client-side interface such as a text editor or a graphical user interface (GUI). The user interface 102 may reside at a client-side computing device 103, such as a laptop or desktop computer. The client-side computing device 103 is coupled to one or more servers 108 through a network 106, such as the Internet.

The server(s) 108 are coupled locally or remotely to one or more databases 110, which may include one or more corpora of libraries including data such as genome data, genetic modification data (e.g., promoter ladders), process condition data, strain environmental data, and phenotypic performance data that may represent microbial strain performance at both small and large scales, and in response to genetic modifications. “Microbes” herein includes bacteria, fungi, and yeast.

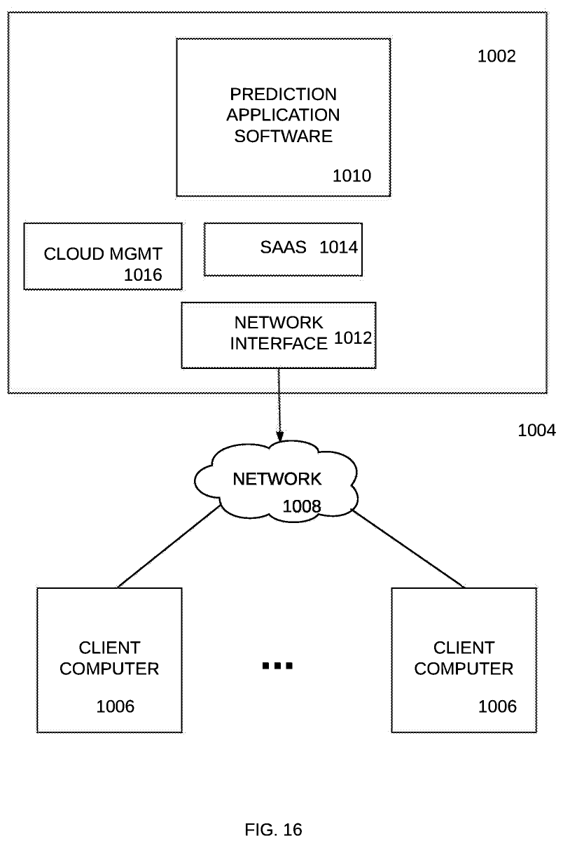

In embodiments, the server(s) 108 include at least one processor 107 and at least one memory 109 storing instructions that, when executed by the processor(s) 107, generates a prediction function, thereby acting as a prediction engine according to embodiments of the disclosure. Alternatively, the software and associated hardware for the prediction engine may reside locally at the client 103 instead of at the server(s) 108, or be distributed between both client 103 and server(s) 108. In embodiments, all or parts of the prediction engine may run as a cloud-based service, depicted further in FIG. 16.

The database(s) 110 may include public databases, as well as custom databases generated by the user or others, e.g., databases including molecules generated via fermentation experiments performed by the user or third-party contributors. The database(s) 110 may be local or remote with respect to the client 103 or distributed both locally and remotely.

The present disclosure provides a robust method for reliably predicting the values of key performance indicators (e.g., yield, productivity, titer) of microbes in larger-scale, low-throughput conditions based on smaller-scale, high-throughput measurements, especially in the technical field of metabolic optimization of organisms for mass-production of chemical targets. Embodiments may employ an optimized statistical model for the prediction. Further, the present disclosure provides a transfer function development tool, which produces the model in a reproducible way, records decisions, and provides a fast and easy mechanism for getting and working with the predicted values.

In this disclosure, a transfer function is a statistical model for predicting performance in one context based on performance in another, where the primary goal is to predict the performance of samples at a larger-scale from their performance at a smaller-scale. In embodiments, the transfer function involves simple, one-factor linear regression between small-scale values and large-scale values, along with optimizations discovered by the inventors. In other embodiments, the transfer function may employ multiple regression.

To build these regression models, embodiments of the disclosure use an input model to summarize the performance of a strain in the high-throughput context (e.g., a plate model), and then use a separate model (e.g., a transfer function) to predict the performance of a strain across multiple runs in the lower-throughput context. The plate model may, for example, be used to model the performance (e.g., yield, productivity, viability) of multiple replicates of the same strain in a 96-well plate. According to embodiments of the disclosure, the prediction engine generates the input model, generates the transfer function, applies the transfer function to the input model output to predict performance, or performs any combination thereof.

The following optimization considerations may be taken into account both in the transfer function and in the summarization models, and in building more complicated, nonlinear machine-learning models for predicting performance in a lower throughput context from performance in a higher throughput context:
accounting for bias due to both the plate and the location on the plate (e.g., row-column location, edge location),
plate characteristics, such as media type/lot, shaker location bias,
process characteristics, like the number of times the glycerol stock used to inoculate wells has been used, and which type of machines (e.g., incubators, fermenters, measurement equipment) were used at both the lower and higher-throughput steps,
sample characteristics (such as cell lineage or presence/absence of known genetic markers)

Approaches for building a robust and reliable transfer function for accurately predicting key performance indicators at larger scale based on smaller-scale high-throughput measurements are presented below, along with a transfer function development tool that records some decisions and makes the process reproducible and fast.

This disclosure first presents a basic linear model according to embodiments of the disclosure. The disclosure then presents optimizations implemented algorithmically according to embodiments of the disclosure. According to embodiments, the transfer function development tool includes an infrastructure to implement further optimizations after the data is in an ingestible format. The following examples are based on the problem of predicting bioreactor (larger-scale, lower-throughput) productivities (g/L/h) and yields (wt %) of an amino acid based on titers of the amino acid at 24 and 96 hours, respectively, in 96-well plates (smaller-scale, higher-throughput) for individual strains.

The Basic Transfer Function: Plate-Tank Correlation

The most basic form of the transfer function is a single-factor linear regression of the form y=mx+b, where x is the value obtained in small-scale, high-throughput screening, y is the value obtained in large-scale, low-throughput screening, and m and b are the slope and y intercept, respectively, of the fit line. Embodiments may also employ multiple regression to predict dependent variable y based on multiple independent variables xᵢ. The correlation between a single x and the y value at the two scales can be used as a measure of how effective this basic approach is; thus it may be called the “plate-tank correlation.”

Even this basic form of the transfer function incorporates an inventive optimization. Instead of simply using the mean performance of a strain to obtain a single value for the strain from the high-throughput screening to correlate to the lower-throughput values, embodiments of the disclosure employ a linear model that corrects for plate location bias, among other factors. Other embodiments employ non-linear models, and account for other aspects of the plate model.

The plate-tank correlation (i.e., transfer) function not only predicts performance of samples that have not been tested at a lower-throughput, larger scale. It also may be used to assess the effectiveness of the plate model. The plate model is a collection of media and process constraints designed to make the values obtained at small-scale in high-throughput as predictive as possible of the values obtained at large scale. The correlation coefficient of the plate-tank correlation function indicates, among other things, how well the plate model is fulfilling its purpose. The plate model may incorporate, but is not limited to, physical features (which may function as independent variables in the plate model) such as:
media formulation and preparation (e.g. media lots)
diluent type
inoculation volume
labware
shaking time, temperature and humidity

In embodiments of the disclosure, the plate-tank correlation function is used to optimize the plate model. In embodiments the plate model mimics the microbial fermentation process at tank scale—to physically model tank performance via implementation in the plates.

Plate Model

The performance of a strain in the high-throughput context (e.g., in a small-scale, plate environment) may be determined via a Least Squares Means (LS-Means) method, according to embodiments of the disclosure. LS-Means is a two-step process by which first a linear regression is fit, and then that fit model predicts the performance over the Cartesian set of all categorical features, and the mean of all numerical features. The features of the model relate the physical plate model to a statistical plate model, and describe conditions under which that experiment was conducted, and include the optimizations listed above (e.g., location on the plate, plate characteristics, process characteristics, sample characteristics).

The model form of the first step is:

titerᵢ=β_{s[i]}+Σ_{fXf[i]}

There is an inferred additive coefficient, βₛ, for the strain's effect (titer in this example), and then each additional feature used in the model. The first term βₛ is the effect (here, titer) of the strain replicate indexed by i. Then each additional term β_{f} is the weighting assigned to feature, f, (e.g., plate location) and x_{f[i]} is the value of the feature for the strain replicate indexed by i.

As an example, one such model might be:

titerᵢ=β_{s[i]}+βₚₗₐₜₑ plateᵢ

In this model, the feature is the particular plate on which the strain is grown. This model includes a coefficient βₚₗₐₜₑ for each strain and each plate indexed by i in the particular experiment. The model may be fit using ridge regression with a penalty to improve numerical stability.

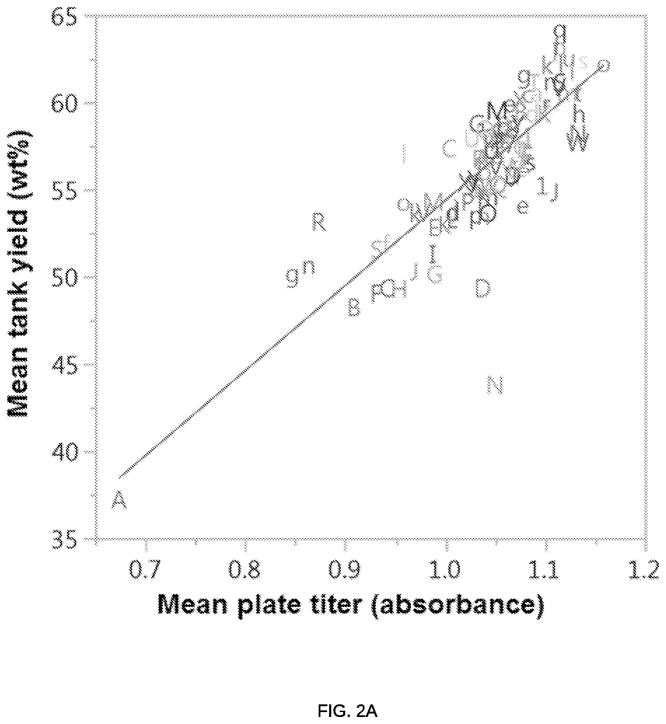

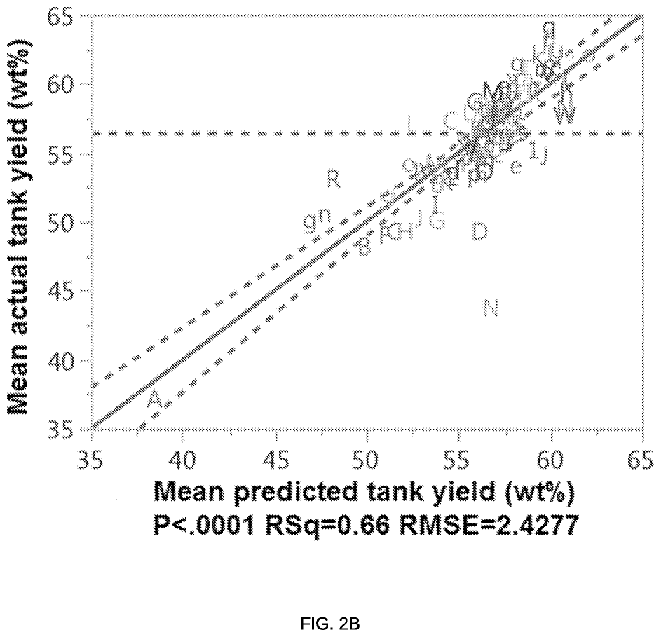

The second step again takes all possible combinations of the factors (e.g., particular plate and location on the plate for all strains) and makes predictions on those synthetic values using the plate model equation to simulate what would occur in the event a strain was run in each scenario, and finally the mean performance of scenarios by strain is taken. This is the final point estimate associated with the plate performance (e.g. the x-axis plate performance value in FIG. 2A), and that is correlated with a summary of tank performance (e.g. the y-axis tank performance value in FIG. 2A).

An example of a correlation according to embodiments of the disclosure is shown in FIG. 2A. FIG. 2A illustrates a comparison of measured bioreactor (tank, larger scale) vs. plate (smaller scale) values for individual strains. The dataset includes high-throughput measurements (using the plate model to determine yield), and associated bioreactor measurements (e.g., yield) for producing an amino acid. Average plate titers (incorporating estimated plate bias) per strain are on the x-axis, and average bioreactor (e.g., tank, fermenter) yields (wt %) per strain are on the y-axis. Each point (letter) corresponds to a single strain.

For purposes of prediction, such plots may be examined in terms of how well the model's predicted performance matches up with the actual performance, which for the simple case shown in the figure is the regression plot with a rescaled x-axis. FIG. 2B illustrates a comparison of actual yield values to simple linear predicted yield values for a bioreactor (tank). The dotted horizontal line is the global mean of actual tank values, and the dotted diagonal lines represent a 95% confidence interval of the actual location of the fit line. Predicted P, RSq, and RMSE are the primary metrics of model performance here, with Predicted P being the P-value of the fit, RSq being the R² of the correlation, and RMSE being the root mean squared error of the predictions. Of these, RMSE is the most useful for optimization purposes, since it is the most direct measure of prediction accuracy.

Optimizations

Outliers

In examining the plots above, some strains behave very differently from the rest and are spatially isolated. These outliers can be classified into two types: Type 1 outliers that represent extreme values in performance, y axis, e.g., yield, and Type 2 outliers that represent, otherwise referred to as “high leverage points” that represent extreme values in the x axis. Type 1 outliers are those strains that are far away from the fit line; i.e., they are predicted poorly (the strain labeled N in the lower right quadrant of FIG. 2B is an example). Such strains affect the fit of the model and can impair predictivity for all other strains while still being poorly predicted themselves. One optimization is to remove such strains to improve the overall predictive power of the model. Another optimization is to add factors to the transfer function model, or to the model that summarizes the strain performance at the higher-throughput level (e.g., plate model incorporating plate location bias, or genetic factors).

Type 2 outliers are those that are on or close to the fit line but still distant from other strains (the strain labeled A in the lower left corner is an example in FIG. 2B). Distance can be measured in a number of ways including: distance from the centroid of the other strains, or distance to the nearest other strain. Type 2 outliers exert outsize influence on the simple linear model. The purpose of the model is to predict, as accurately as possible, the performance of the remaining strains. Thus, embodiments of the disclosure optimize with regard to Type 2 outliers by removing them (in conformance with general statistical practice), or alternatively, by optimizing the model by adding predictive factors.

In the case of optimizing by removal of the outlier, embodiments of the disclosure provide at least two approaches to labeling a strain as an outlier to be removed:

The first is on the basis of the strain appearing repeatedly as an outlier and on having a meaningful rationale based on the unusual characteristics of the strain or its performance at a larger scale to exclude it as not representative of the bulk of strains. For instance, the A strain in FIG. 2B is a progenitor of the other strains in the model, but genetically and in performance at scale rather distant from them. The N strain has a modification known to give good results in the plate but to fails to consume enough glucose at larger scales.

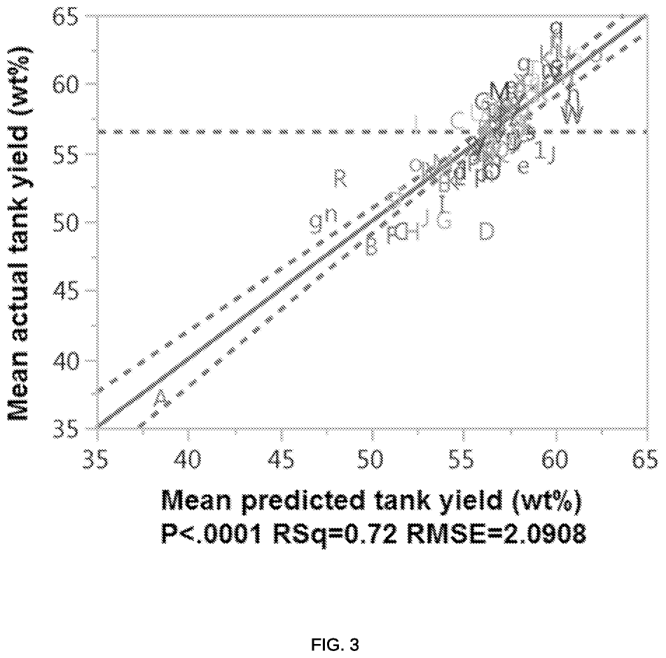

The second outlier-labeling method is to assign a “leverage metric” to each strain and consider it an outlier if the change in the metric due to removal of the strain exceeds a predefined cutoff (“leverage threshold”). For instance, the leverage metric may represent the percentage difference in RMSE with and without the strain in the model, and the cutoff may be a 10% improvement. In this case, the results of removing the N strain are depicted in FIG. 3.

FIG. 3 is a plot equivalent to that of FIG. 2B, except with Type 1 outlier strain N removed. Removing the N strain decreases the RMSE from 2.43 to 2.09, or 14%, which is higher than the currently used cutoff of 10%. Thus, the prediction engine would identify the outlier for removal.

Care should be taken in removing outlier strains (e.g., setting the outlier cutoff too low) because of the danger of overfitting, i.e., building a model that predicts a small subset of strains very well but does poorly when used on the broader population. One way to protect against this is to use a cut-off that is weighted by the number or fraction of candidate strains in the model. For instance, if the base cutoff is 10% and there are 100 strains that could be included the model, the cutoff for removing the first strain may be 0.1/0.99, the cutoff for removing the second strain could be 0.1/0.98, the cutoff for the third 0.1/0.97, etc.

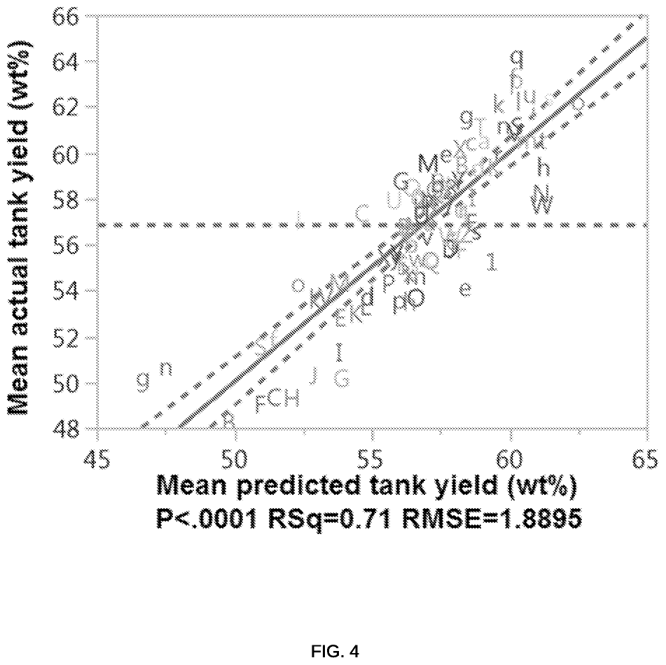

After removing one Type 2 outlier and four Type 1 outliers, the fit of FIG. 3 becomes as shown in FIG. 4. FIG. 4 is a plot equivalent to that of FIG. 2B, except with four Type 1 outliers and one Type 2 outlier removed. Note that RSq and RMSE are both improved in FIG. 4, by approximately 6% and 21%, respectively, relative to the model in FIG. 2B.

Genetic and Other Factors

Genetic or other characteristics of the samples (including process aspects, such as the lot number of the media used for growing the strains) can also be useful for improving predictive power as factors in the transfer function, especially given that a high-throughput plate model alone is unlikely to completely recapitulate the conditions that samples will be subjected to at a larger scale. In the case of metabolic engineering, in particular, it is impossible to reproduce conditions in a five-liter or larger bioreactor, such as the effects of fluid dynamics, shear stresses, and diffusion of oxygen and nutrients, in 200-μL wells in a plate. Work towards improving the physical plate model based on factors such as media composition, method of media preparation, compounds measured, and timing of measurements has downsides in being time-consuming and expensive, and possibly making it difficult to compare samples run under a new plate model to those run under the old. Thus, embodiments of the disclosure identify and make use of other predictive factors of the plate model to improve predictions. Some of those other factors, according to embodiments of the disclosure, include:
accounting for bias due to location of strain on a plate
plate characteristics, like media type/lot, shaker_location bias
process characteristics, such as the number of times the glycerol stock used to inoculate wells has been used and which type of machines were used at both the lower and higher-throughput steps
sample characteristics (such as cell lineage or presence/absence of known genetic markers)

The inventors have found genetic factors, in particular, to be useful in improving the transfer function for metabolically engineered strains—for example, incorporating information about changes that lead to differences in gene regulation.

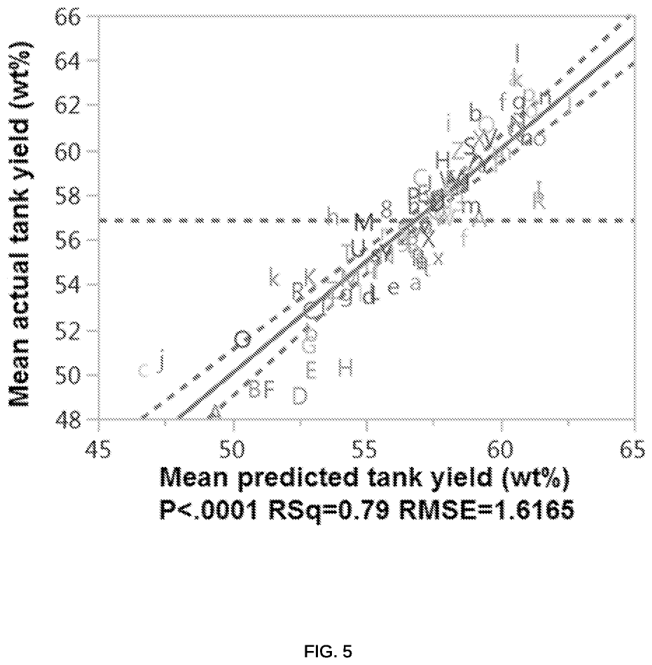

FIG. 5 depicts the result of applying a correction to all the strains in FIG. 4 based on whether or not they have a certain genetic modification (e.g., a start-codon swap in a particular gene). As an example, for a multiple regression transfer function model, the adjustment/correction accounting for the presence or absence of the start-codon swap may take the form of adding a performance component mᵢxᵢ or a performance component mⱼxⱼ, respectively, to the mean tank yield performance of the strains predicted by the transfer function. (Note that the weight m may take on negative values.) In embodiments, mᵢ may take on a single value, and x is +1 or −1 depending upon whether the modification is present or not, respectively. In other embodiments, mᵢ may take on a single value, and x is +1 or 0.

FIG. 5 is equivalent to FIG. 4, except it includes a correction factor for the presence or absence of a start codon swap in the aceE gene. This correction increases the RSq from 0.71 to 0.79 and decreases the RMSE from 1.9 to 1.6 (16%).

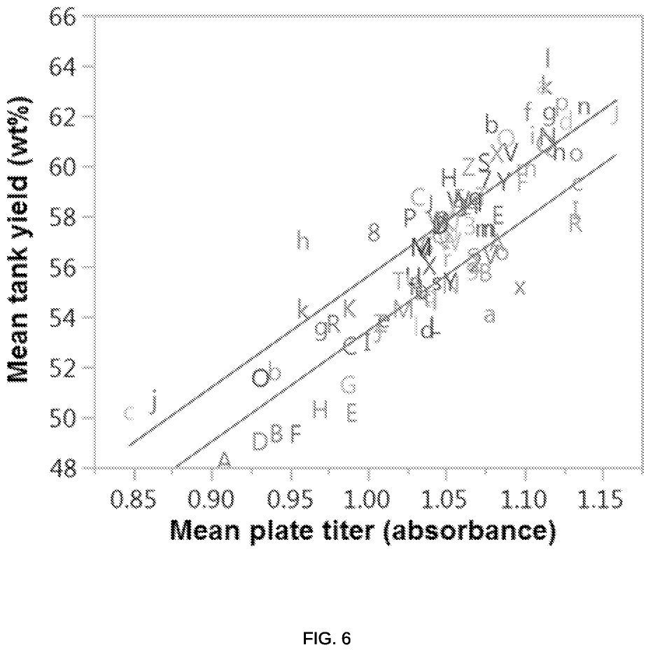

FIG. 6 is a regression plot of the model shown in FIG. 5. The regression plot (FIG. 6) shows that essentially two regression lines are used, depending on whether the modification is present (upper line) or absent (lower line).

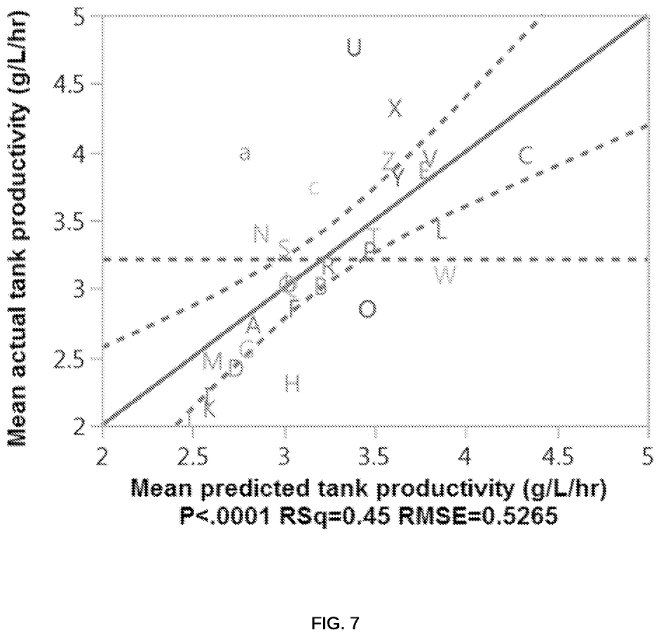

FIG. 7 illustrates a productivity model without correction for genetic factors. The results of correcting for genetics are even more striking in the productivity model. Without correcting for a genetic change that the plate model fails to recapitulate (e.g., a promoter swap), the model is as shown in FIG. 7.

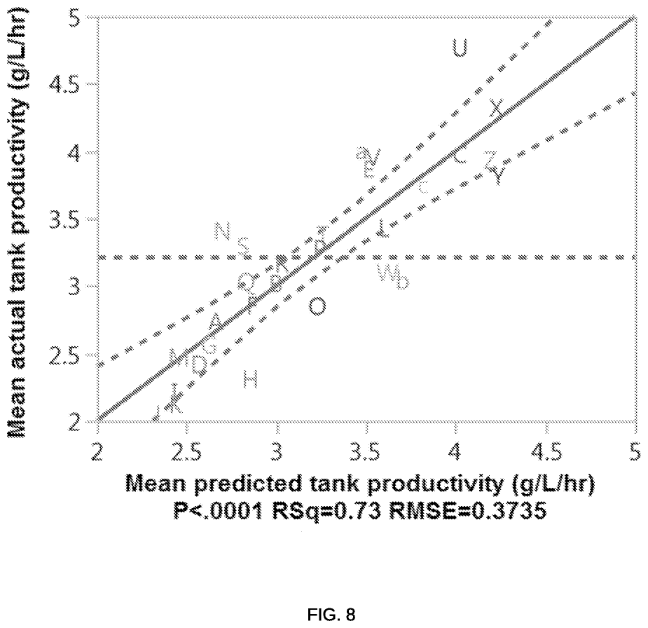

Including the correction for the presence or absence of this modification yields the model shown in FIG. 8. FIG. 8 illustrates the productivity model of FIG. 7 after correction for a genetic factor (e.g., a particular promoter swap). A promoter swap is a promoter modification, including insertion, deletion, or replacement of a promoter.

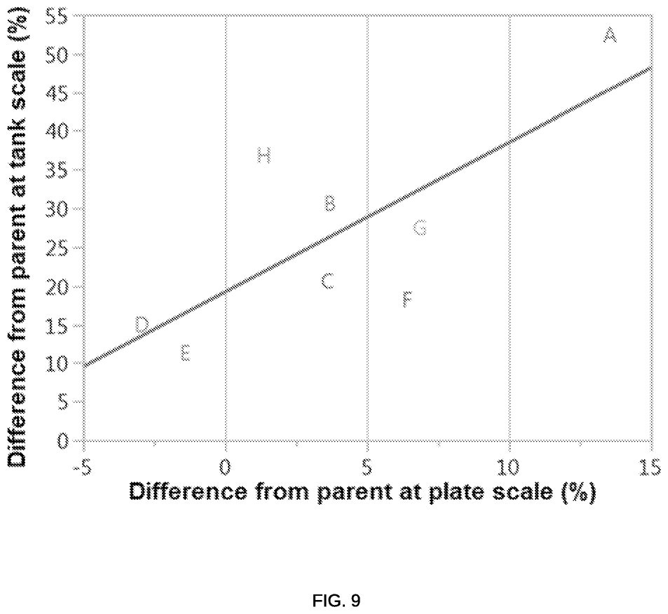

Including this factor in the model (e.g., multiple regression model) increases RSq from 0.45 to 0.73 and reduces RMSE from 0.53 to 0.37 (30%), which is an impactful increase in predictive power. In fact, examining the improvement in plate performance (“hts_prod_difference”) versus the improvement in bioreactor (tank) performance (tank_prod_difference) for strains harboring this modification (with two outliers removed) and fitting them to a line yields FIG. 9.

FIG. 9 illustrates improvement in the high-throughput productivity-model performance (x-axis) versus improvement in actual productivity in low-throughput bioreactors (e.g., tanks) (y-axis) for strains harboring the same promoter swap as in FIG. 8.

The equation of the fit line is 19+1.9*hts_prod_difference, meaning that a strain harboring this change that is indistinguishable from its parent in the plate model can be expected to perform approximately 20% better than its parent at scale, a major improvement that the plate model alone cannot accurately predict. Even strains that the plate model alone predicts will be worse at the plate level than parent (like D and E in the plot of FIG. 9) are in fact much better than parent at tank scale. Including a factor for this change in the model accurately predicts these effects in new strains and avoids losing such strains as false negatives.

Groups of genetic factors may also be useful in prediction, as a result of epistatic interactions, in which the effect of two or more modifications in combinations differs from what would be expected from the additive effects of the modifications in isolation. For a more detailed explanation of epistatic effects, please refer to PCT Application No. PCT/US16/65465, filed Dec. 7, 2016, incorporated by reference in its entirety herein.

Another factor is lineage. Lineage is similar to genetic factors in that it is hereditary, but lineage takes into account both the known and unknown genetic changes that are present in a strain compared to other strains in other lineages. Embodiments of the disclosure employ lineage as a factor to build a directed acyclic graph of strain ancestry, and test the most connected nodes (i.e., the progenitor strains that have been used most frequently as targets for further genetic modifications or have the largest number of descendants) for their utility as predictive factors.

Modifications to Transfer Function Output

The simplest way to use transfer function output is to use the output as a prediction of performance at scale. Another approach is to apply the percent change in transfer predictions between parent and daughter strain to the actual large-scale performance of the parent (i.e., prediction=parent_performance_at_scale+parent_performance_at_scale*(TF_output(daughter)−TF_output(parent))/TF_output(parent)), where parent_performance_at_scale is the observed performance of the parent strain at scale (i.e., larger scale), TF_output(strain) is the predicted performance of a strain “strain” due to application of the transfer function, and the daughter strain is a version of the parent strain as modified by one or more genetic modifications. This has the benefit of removing noise associated with the influence of the parent on the daughter's performance at scale, but assumes that such influence exists; i.e., it assumes that the transfer function's error in predicting the daughter's performance will be of approximately the same magnitude and sign as the error in predicting the parent.

Other Statistical Models

The above assumes the transfer function uses simple linear and multiple regression models, but more sophisticated linear models, such as ridge regression or lasso regression, may also be employed in embodiments of the disclosure. Additionally, non-linear models, including polynomial (e.g., quadratic) or logistic fits, or nonlinear machine learning models such a K-nearest neighbors or random forests may be employed in embodiments. More sophisticated cross-validation approaches may be used to avoid overfitting.

### Algorithm Example

In embodiments, the decisions for what samples (strains) to include or exclude as outliers and what potential factors to include to improve predictive power are implemented in an algorithm to ensure reproducibility, explore as many possibilities for improvement as possible, and reduce the influence of subconscious bias. A variety of approaches may be adopted, and an example of one such cyclic/iterative process is presented below, in which the small scale, high throughput environment may correspond to a plate environment, and the large scale, low throughput environment may correspond to a tank environment.
1. Start with a set of strains, using performance measurement(s) (e.g., amino acid titer) as sole factor(s) for developing the predictive model (e.g., linear regression)
   a. These are strains for which actual plate and tank performance data are known.
2. Identify the strain whose removal from the transfer function model most improves RMSE for the model (“the Outlier”).
   a. Alternatively, identify for potential removal from the model the strain that has the greatest prediction error (predicted vs. measured performance for the strain).
3. If the RMSE improvement from removing the strain is greater than a predefined cut-off, proceed to Step 4; otherwise go to Step 10.
4. Identify potential predictive factors that apply to the Outlier that are not present in all other strains currently included in the model (because factors that are equivalent in all strains are not useful for overall predictive power), and are not already included as factors in the model. Optionally, the algorithm may identify factors present in at least one other strain, while still meeting the above conditions.
   a. Factors that are characteristic of the Outlier strain may include, for example, genetic changes known to have been made, lineage (history of strain ancestry), phenotypic characteristics, growth rate.
   b. Note that if a factor is in only one strain, the algorithm may adjust the model to correct for that single strain, but usually modifying the model to account for a single strain may not be an expected objective. Also, if the factor is in all other strains, then it has no predictive value.
   c. Note that embodiments may employ a machine learning model that would automatically perform this function, but that identifying the factors for the model may reduce the resource burden on the machine learning model.
5. If the list from Step 4 is empty, exclude the Outlier from the model and go to Step 2.
6. Otherwise, provisionally apply the factors from Step 4 in the model.
   a. As noted above, embodiments may employ a simple linear regression transfer function such as y=m₁x₁+b, where x₁ is the performance of a strain on the plate, and m₁ is a weight (slope) applied to x₁. In embodiments, the model may be refined by adding weighted factors (regression coefficients) to generate a multiple regression model of the form y=m₁x₁+m₂x₂+ . . . +m_{N}x_{N}+b, where x₁ is the performance of a strain on the plate, the other xᵢ (i≠1) represent factors other than performance x₁, m₁ is a weight applied to x₁, and mᵢ is a weight applied to factor xᵢ. In embodiments, x₁ may represent the output of a plate model. In embodiments, all xᵢ may represent the output of a plate model.
   b. In embodiments, the factors may be added one at a time, and the weighting adjusted, until error (or P value) is reduced by a satisfactory amount before adding the next factor.
7. The algorithm may remove factors (e.g., x values in the multiple regression equation) if the factors do not improve the error of the model by an error threshold or if they have a P-value above a P-value threshold. For example, embodiments of the disclosure may remove particular genetic factors (i.e., genetic modifications known to have been made in the strain) from the regression model (prediction function) if those factors do not improve the error by an error threshold or if they have a P-value above a P-value threshold.
8. According to embodiments of the disclosure, if any remaining genetic factors are part of a group having a high variance inflation factor (e.g., >3, indicative of colinearity between factors), the prediction engine may keep only the genetic factor with the lowest P-value within each group. A high variance inflation indicates a high correlation between factors. Including highly correlated factors would not provide much predictive value and could cause overfitting. According to embodiments of the disclosure, the prediction engine may use variance inflation factor to measure the correlation between factors, and start with removing highly correlated factors until a satisfactory a satisfactory variance inflation factor is reached.
9. If all the genetic changes from Step 4 have been removed at this point, remove the Outlier strain from the model, and return to Step 2.
   a. If the condition is true, the algorithm has determined that the algorithm cannot be satisfactorily improved without removing the Outlier.
10. After iterating through Steps 2-9 or jumping here from Step 3, remove any factors that apply to none or all of the remaining strains. Optionally, remove any genetic factors that only apply to one strain.

The result of the above algorithm may be an improved model with some outliers removed and the model adjusted to account for more factors. The outputs include strains used to develop the model and factors used in the model, along with their weights.

According to embodiments of the disclosure, the prediction engine may compare performance error metrics for a plurality of prediction functions, and rank the prediction functions based at least upon the comparison. Referring to the algorithm above, the prediction engine may compare the predictive performance of models created by different iterations (e.g., different outliers removed, different factors added). According to embodiments, the prediction engine may compare the predictive performance of models created by different techniques, e.g., ridge regression, multiple regression, random forest.

Embodiments of the disclosure test new versions of the transfer function and monitor its performance by measuring actual performance of the strain at large scale. A new transfer function's predictions may be back-tested against other versions of the transfer function and compared in performance on historical data. Then the transfer function may be forward-tested in parallel with other versions on new data. Metrics of performance (such as RMSE) may be monitored over time, so that improvements may be made quickly if performance begins to fall off. (Similar processes can be used to improve and monitor the plate model, and the two processes can also be combined to include a decision point as to whether efforts toward improvement should focus on the transfer function or the plate model.)

In embodiments, if the transfer function fails to accurately predict strain performance at the bioreactor scale, physical adjustments may be made to the physical plate cultivation model. As with adjustments to the parameters/weights of the mathematical model, physical changes to the physical plate model may be made based on the phenotype of interest. Several changes may be made and evaluated to determine which physical plate model(s) yield the best transfer function. Examples of changes include, but are not limited to, media composition, cultivation time, compounds measured, and inoculation volume.

### EXPERIMENTAL EXAMPLES

The following two examples show use of embodiments of the disclosure to produce different products of interest in different organisms.

### Example 1

When fitting a statistical model for predicting performance of microbes at a larger scale (e.g., tank) based on a smaller scale (e.g., plate), embodiments of the disclosure use multiple metrics as well as standard statistical techniques for fitting the model. In these experiments, the prediction engine uses multiple plate measurements per plate to derive a predictive function, and the plate values are based on statistical plate models that are themselves based on raw, measured physical plate data. This Example 1 covers one main product, a polyketide produced by a Saccharopolyspora bacterium.

In the following discussion, embodiments of the disclosure make use of the standard adjusted R², root mean squared error (RMSE) for a set of test strains, and a leave one out cross validation (“LOOCV”) metric.

RMSE: A set of strains, the training strains (marked as “train”), were used to fit the model. Then the prediction engine screened many new strains in plates (not the strains used to train the model), and promoted a subset of those strains to tanks (i.e., selected those strains with good statistics to be generated in tanks at the larger scale). The prediction engine computed

$$RMSE = \sqrt{\sum \frac{{( t a n {k}_{a c t u a l} - t a n {k}_{predicted} )}^{2}}{n}}$$

for this set of test strains, where n is the number of test strains, and the variable tank is the performance metric of interest (e.g., yield, productivity) at tank scale.

LOOCV: According to embodiments of the disclosure, for any new model, according to LOOCV the prediction engine iterated through the set of training strains. At each step, the prediction engine removed a strain from the training data, fitted the model using the remaining training data, and computed the RMSE for the removed, former training strain as a test strain (see previous discussion of RMSE). The prediction engine set RMSEᵢ to be the RMSE with the i^{th} strain removed. The prediction engine then computed the mean of this set of RMSE values so

$$LOOCV = \frac{{Σ}_{i} {RMSE}_{i}}{m}$$

where m is the total number of strains in the training set.

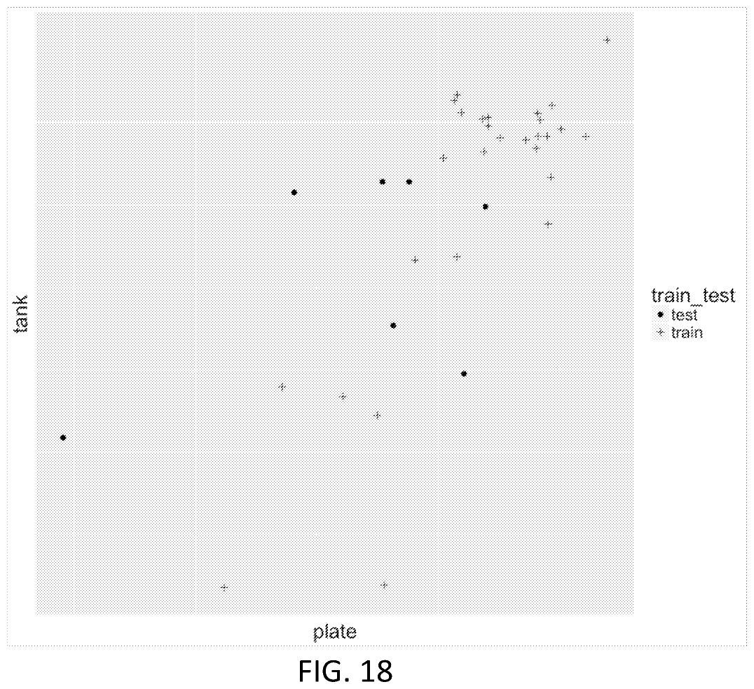

FIG. 18 is a graph of the plate vs. tank values for the primary metric of interest. The figure shows a reasonable linear relationship. If the prediction engine fits the simple linear model tank=b+m₁*plate_value₁ on the microbes marked as train, where b=−3.0137, m₁=0.0096 and plate_value₁ is a polyketide value in mg/L processed by the statistical plate model, then the adjusted R\hat{ }2 is 0.65, the leave one out CV is 2.65, and the RMSE of the test set is 5.2152.

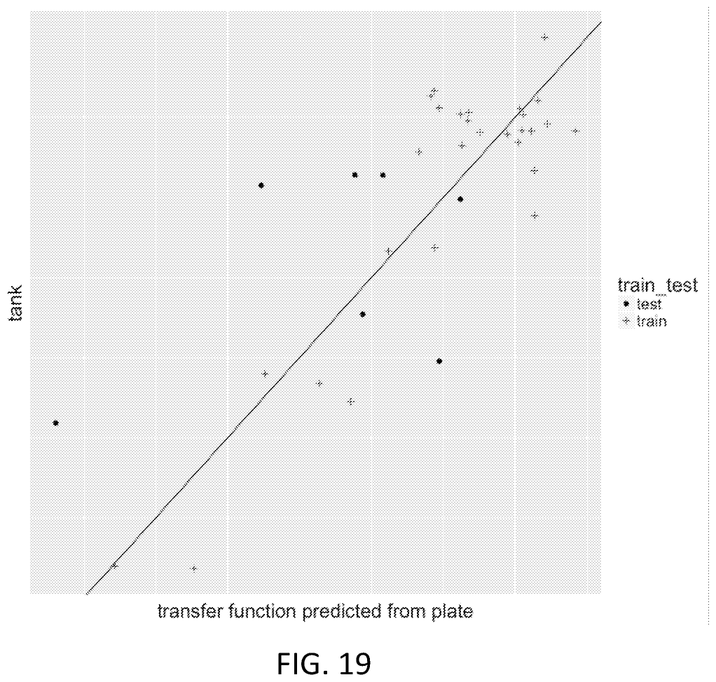

If the prediction engine instead fits the linear-regression model tank=b+m₁*plate_value₁+m₂*plate_value₁*plate_value₂, where b=0.7728, m₁=0.0325, m₂=0.0000646, and both plate_values are for two different polyketides (in mg/L) processed by the statistical plate model, the prediction engine provides a much more predictive transfer function, as shown in the FIG. 19. Note that the plate values plate_value₁, plate_value₂, etc. represent assays on the same plate, and can be the same or different assays on the plate, e.g., all product of interest assays (e.g., yield), or instead product of interest and another assay, such as biomass or glucose consumption. According to embodiments of the disclosure, the plate value or tank value may represent a mean amount of a given value for the plate or tank, respectively.

This transfer function has a LOOCV of 2.25 an adjusted R² of 0.77, but most importantly, the RMSE on the test set drops to 4.36.

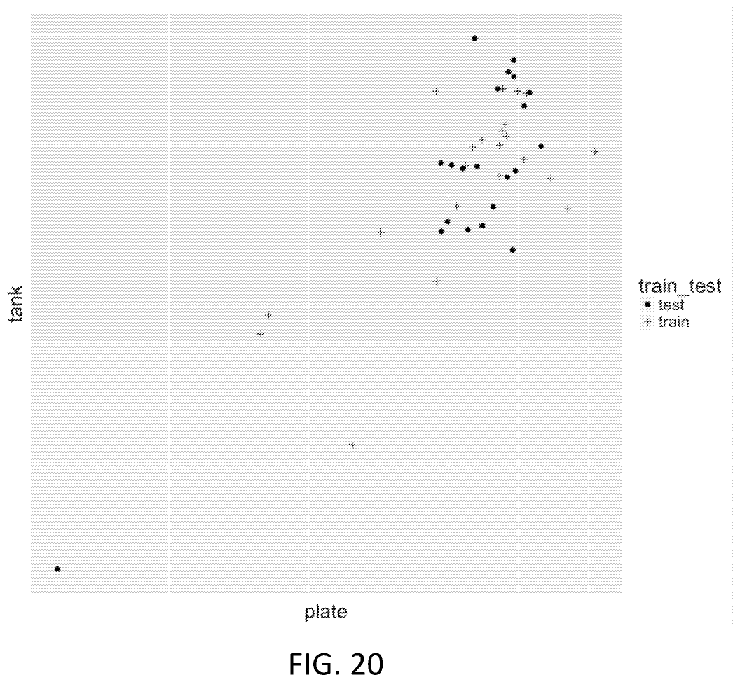

After getting more data and updating the plate and tank data, the plate vs. tank values for the primary metric of interest are as shown in FIG. 20.

The simple linear model tank=b+m₁*plate_value₁, where b=2.735544, m₁=0.009768, had mixed results for these data. The LOOCV is 3.16 and the adjusted R² is 0.49. The LOOCV is worse and the adjusted R² much worse than the previous iteration, but the RMSE on the test set goes down significantly to 2.8.

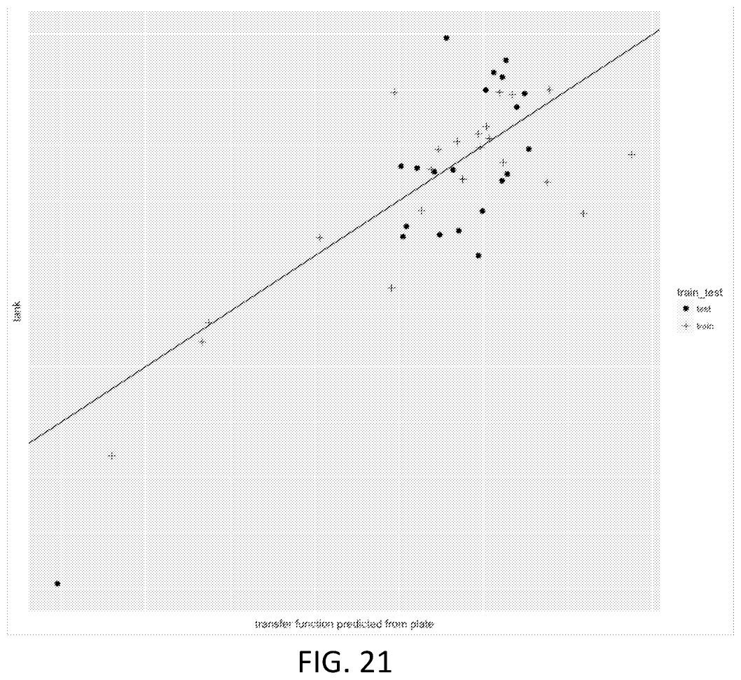

The prediction engine was run with a weighted least squares model of the form above: tank=b+m₁*plate_value₁+m₂*plate_value₁*plate_value₂, but with regression coefficients mᵢ dependent upon the number of replicates at tank scale, where b=6.996, m1=0.01876, and m2=0.000237 with the same two polyketides (as before in mg/L). Here, an improved model was obtained by all metrics except the LOOCV, as shown in FIG. 21. (The plate values were provided by a statistical plate model.) These statistics are LOOCV=3.14, adjusted R\hat{ }2=0.79, and RMSE on the test set=2.99. As background to factoring the number of tank-scale replicates into the weights mᵢ, the weight vector is determined using ordinary least squares by solving y=Xm+e (here y is a vector of the observed tank values and X is a matrix of the plate values). The weight vector is computed as m=(X^{T}X)⁻¹X^{T}*y. This formulation assumes the variances of the errors (which are random variables) are all the same. However, this assumption generally does not hold in experiments—the number of replicates in the tanks greatly affects variance calculations, and strains typically do not have equal variances, so their errors in this formulation also will not be equal. Allowing the errors to be different, then when we fit the model above, we instead get m=(XTWX)⁻¹X^{T}Wy where W is a diagonal matrix and the diagonal entries are the “weights”. The weights are interpreted as being wᵢ=1/sigmaᵢ², where sigmaᵢ² is the variance of the i^{th} error. This effectively means that more weight (more influence in the fit too) is given to observations with small variance, and less weight (influence) is given to observations with high variance. According to embodiments of the disclosure, we take wᵢ=the number of tank replicates, and in that way strains that have more observations have more weight in the fit because less error overall is expected in the observations of those strains.

In another trial, the prediction engine produced another prediction (transfer) function, where the time the assays were taken was changed and a new set of training strains was used. There is no test data for this function yet. Using the previous weighted least squares approach for the same polyketides as above with the formula tank=b+m₁*plate_value₂+m₂*plate_value₂*plate_value₃, where b=−4.482, m₁=0.05247, m₂=0.0001994, the adjusted R² jumps to 0.93, but the LOOCV is high at 7.44, suggesting there are some high leverage points.

An additional plate value for this model was tested, still using weighted least squares but using the formula b+m₁*plate_value₂+m₂*plate_value₂*plate_value₃+m₃*plate_value₄, where b=−1.810, m₁=0.0563, m₂=0.0001524, m₃=0.5897, plate_value₂ and plate_value₃ are mg/L metrics for the same two polyketides as above, and plate_value₄ is biomass measured in optical density (OD600). The LOOCV dropped to 6.22, still higher than before, but much lower than the previous value and the adjusted R\hat{ }2 is now 0.95. Of course, the true test of this transfer function is testing its predictive power on new strains.

### Example 2

This second example mirrors some aspects of Example 1 in that a set of transfer functions were fit that successively included additional plate measurements per plate (e.g., different types of measurements such as yield, biomass) to try to fit a finer estimate of tank performance. This Example 2 covers one main product, an amino acid produced by a Corynebacterium. Additionally, this example shows the case of applying the transfer function to a different tank variable measurement (here dubbed “tank_value₂”).

One Tank Measurement, Multiple Plate Measurements

Model 1

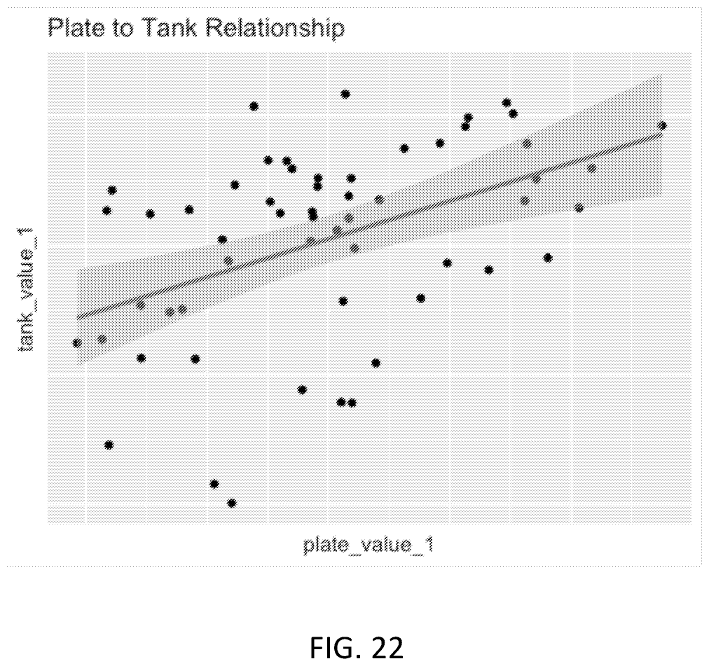

In the first model we fit a simple model that assumed tank_value₁˜1+plate_value₁, according to embodiments of the disclosure. Note that “˜” refers to a “function of, according to a predictive model, such as linear regression or multiple regression.” The underlying plot of FIG. 22 shows the relationship between values of the plate value (represented in the statistical plate model) against the observed tank value.

As can be seen from the plot, when modeling the tank value output on one of the plate metrics, there is potentially a linear relationship between the two.

Taking another step, the prediction engine conducted LOOCV (leave-one-out cross validation) to get the performance of the model by training on every strain except for one, then testing the fit against that one value. The LOOCV score, then, is the average of all the test metrics taken as each data point is removed.

Doing so resulted in the following performance:

| ## | RMSE | MAE |
|---|---|---|
| ## 1 | 3.262872 | 2.532292 |

In particular, with RMSE, the prediction engine computed the ratio of RMSE to the mean tank performance to get a sense of the magnitude of the error relative to the average outcome:

## [1] 5.416798

This result indicates that there's about 5% error on the estimate relative to the average values of the tank performance.

Model 2

Now that the inventors had obtained a baseline, they added to the model another measurement from the same plate to compare performance, resulting in a predictive function of the form tank_value₁˜plate_value₁+plate_value₂, with the following statistics:

| ## | RMSE | MAE |
|---|---|---|
| ## 1 | 3.376254 | 2.59808 |

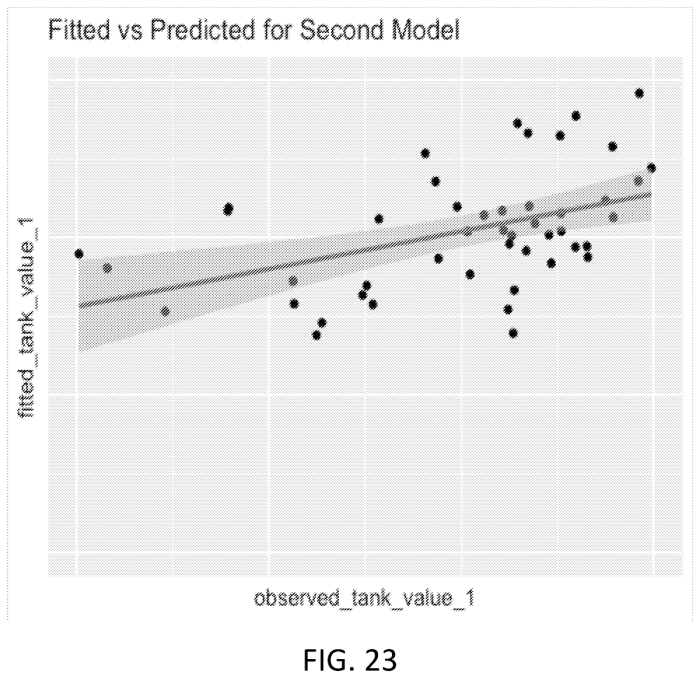

Performance appears slightly worse in this case, as the RMSE and the MAE are a bit higher. See FIG. 23.

Model 3

Finally, in a third example of this process the inventors added yet another factor, such that the model is tank_value₁˜plate_value₁+plate_value₂+plate_value₃.

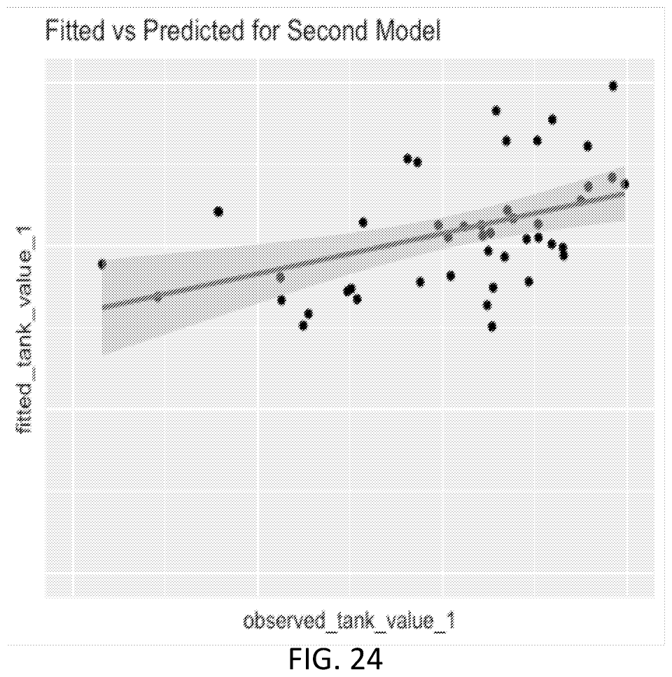

Referring to FIG. 24, this provides a slightly better fit than the first model, as the LOOCV using an RMSE metric is slightly lower for this model.

| ## | RMSE | MAE |
|---|---|---|
| ## 1 | 3.224997 | 2.51152 |

Accordingly the relative percent error is slightly lower than the original model.

## [1] 5.353921

Multiple Tank Measurements

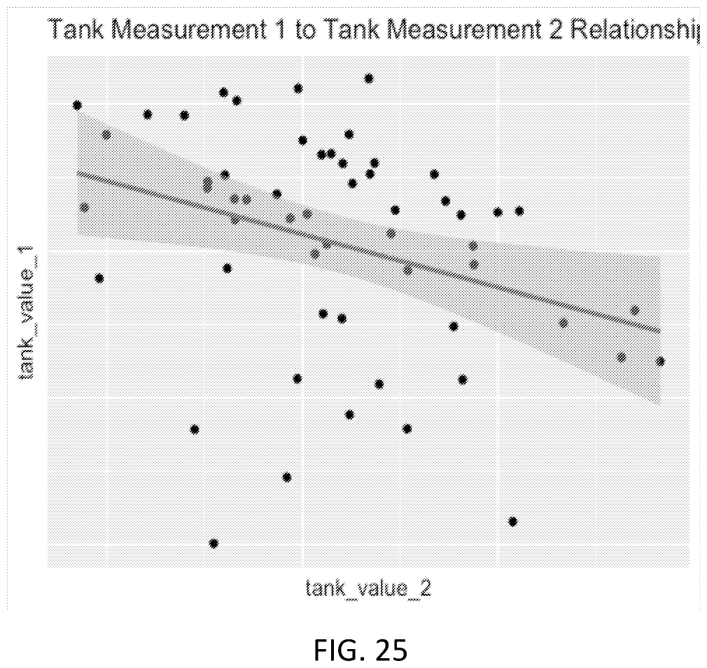

As referenced, the transfer function can be applied to predict multiple outcomes for the same tank. For example, the prediction engine fit a model previously of the form tank_value₁˜plate_value₁, but in another trial the prediction engine fit another model to a different output (e.g., yield instead of productivity): tank_value₂˜plate_value₁. FIG. 25 plots two measured tank values against each other.

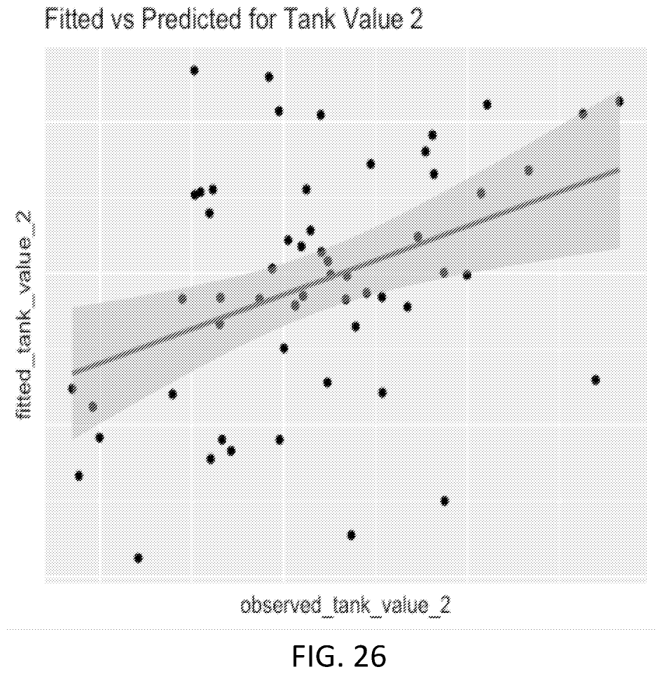

Referring to FIG. 26 the prediction engine fit a model of the form tank_value₂˜plate_value₁, where the observed measurements for tank_value₂ are known a priori to be much more variable than those for tank_value₁. Thus, one would expect that, a priori, the metrics for this model will not be as good as those above. The prediction engine fits this model resulting in an RMSE and MAE of:

### ##RMSE MAE

##1 0.6315165 0.501553

Compared the RMSE to the actual value provides a sense of the magnitude of the error:

##[1] 19.88434

If desired, the iterative approach may be repeated as described above to add or remove features based on the model's LOOCV performance.

Predictive Model Accounting for Microbial Growth Characteristics

The section “Other statistical models” herein refers to a variety of predictive models. According to embodiments of the disclosure, the prediction engine accounts for microbial growth characteristics. According to embodiments of the disclosure, the prediction engine combines multiple plate-based measurements into a few microbially relevant parameters (e.g., biomass yield, product yield, growth rate, biomass specific sugar uptake rate, biomass specific productivity, volumetric sugar uptake rate, volumetric productivity) for use in transfer functions.

According to embodiments of the disclosure, a transfer function is a mathematical equation that predicts bioreactor performance based on measurements taken in one or more plate-based experiments. According to embodiments of the disclosure, the prediction engine combines the measurements taken in plates into a mathematical equation, e.g.:

PBP=a+b*PM1+c*PM2 . . . n*PMn

in which:
PBP=predicted bioreactor performance (e.g., y in other examples herein),
PMi=the ith plate data variable (e.g., first scale performance data variable xᵢ in other examples herein), which can be a measurement or a function of measurements, such as a combination of measurements or a statistical function of measurements (e.g., a statistical plate model), and
a, b, c, . . . n, may be represented as mᵢ as in other examples herein

The above equation is a linear equation. According to embodiments of the disclosure, the prediction engine may also employ transfer functions of the following form:
quadratic equation (e.g., PBP=a+b*PM1\hat{ }2+c*PM2\hat{ }2)
interaction equation (e.g., PBP=a+b*PM1+c*PM2+d*PM1*PM2)
a combination of different equations

According to embodiments of the disclosure, the prediction engine employs a transfer function that accounts for microbial growth characteristics. Combining linear with quadratic, polynomial or interaction equations can result in many parameters (e.g., a, b, c, d, n) to fit. In particular when only few “ladder strains” (set of diverse strains that have different and known performance) exist against which to calibrate the model, this can result in overfitting of the data and poor predictive value

Thus, based on microbial growth dynamics, the prediction engine may employ a mathematical framework that combines multiple measurements into a few microbially relevant parameters (e.g., biomass yield, product yield, growth rate, biomass specific sugar uptake rate, biomass specific productivity, volumetric sugar uptake rate, volumetric productivity) using selected subtractions, divisions, natural logarithms and multiplications between measurements and parameters. (This approach is discussed further with respect to a prophetic example.)

In general, the prediction engine of embodiments of the disclosure considers two types of plate-based measurements:
Start & end-point measurements, which can be used to assess conversion yields
Mid-point measurements, which can be used to assess conversion rates and yields

Start & End-Point Measurements and Calculation of Microbial Parameters

Typical Measurements:

Cx—Biomass concentration (e.g., measured by optical density (“OD”))

Biomass concentration at the start point of the main culture can be either:
Deduced from measuring biomass at the end point in a seed culture, and correcting for transfer volume and main culture volume, i.e., biomass concentration at start point of main culture=biomass concentration at end point of seed culture*(seed to main transfer volume)/(main start volume). A seed culture includes the workflow to revive a set of strains from a frozen condition. The “main” culture includes the workflow to test the performance of the strains.
Estimated as constant from development experiments (e.g., when all strains have a starting biomass concentration of OD 0.1-0.15, the average could be taken as a proxy). The biomass concentration at the end of cultivation (growing a microorganism under particular conditions) is typically much higher than at the start, and the biomass concentration at the start can mathematically be left out of some equations (e.g., if final biomass concentration is more than ten times higher than initial concentration, when measuring biomass yield).

Cp—Product Concentration

Note: the same measurements and calculations for product concentration can be performed for byproducts of interest.

Product concentration at start can be either:
Deduced from measuring product at end in seed culture, and correcting for transfer volume and main culture volume, i.e., product concentration at start of main culture=(product concentration at end of seed)*(transfer volume)/(main start volume)
Estimated as constant from development experiments (e.g., when all strains have a starting product concentration of 0.1-0.15 g/L the average could be taken as proxy). Please note that the product concentration at the end of cultivation is typically much higher than at the start, and that the product concentration at the start can mathematically be left out.

Cs—Sugar Concentration

Sugar concentration at the start is a known parameter from medium preparation.

Sugar concentration at the end of cultivation is often zero, but can be measured, if needed.

Calculation of microbially relevant parameters:

Biomass yield (Ysx, gram cells per gram sugar)

$$Ysx = \frac{Cx ( end ) - Cx ( start )}{Cs ( start ) - Cs ( end )}$$

i.e., biomass yield=(biomass concentration at end−biomass concentration at start)/(sugar concentration at start−sugar concentration at end)

Product (or byproduct) yield (Ysp, gram product per gram sugar)

$$Ysp = \frac{Cp ( end ) - Cp ( start )}{Cs ( start ) - Cs ( end )}$$

Product (or byproduct) yield=(product concentration at end−product concentration at start)/(sugar concentration at start−sugar concentration at end)

Mid-Point Measurements & Calculation of Microbial Parameters

Typical Measurements:

Time, e.g., t1 and t2

Note: t1 can be start of main cultivation. See above for how to estimate Cx and Cp at the start of cultivation

Cx—Biomass Concentration (e.g. Measured by Optical Density)

According to embodiments of the disclosure, biomass concentration at t1 or t2 is measured, if possible given broth composition

Cp—Product Concentration

According to embodiments of the disclosure, product concentration at t1 and t2 is measured

Cs—Sugar Concentration

According to embodiments of the disclosure, sugar concentration at t1 or t2 is measured

Sugar concentration at start is a known parameter from medium preparation

Calculations

Biomass Yield (Ysx, Gram Cells Per Gram Sugar)

$$Ysx = \frac{Cx ( t 2 ) - Cx ( t 1 )}{Cs ( t 1 ) - Cs ( t 2 )}$$

i.e., biomass yield=(biomass concentration at t2−biomass concentration at t1)/(sugar concentration at t1−sugar concentration at t2)

Product Yield (Ysp, Gram Product Per Gram Sugar)

$$Ysp = \frac{Cp ( t 2 ) - Cp ( t 1 )}{Cs ( t 1 ) - Cs ( t 2 )}$$

i.e., product yield=(product concentration at t2−product concentration at t1)/(sugar concentration at t1−sugar concentration at t2)

Exponential growth rate (mu, per hour)

$$m u = \frac{ln ( \frac{C x ( t 2 )}{Cx ( t 1 )} )}{( t 2 - t 1 )}$$

i.e., mu=ln(biomass concentration at t2/biomass concentration at t1)/(time of t2−time of t1)

based on exponential growth: Cx(t2)=Cx(t1)*exp(mu*(t2−t1))

Biomass specific sugar uptake rate (qs, gram sugar per gram cells per hour)

$$q s = \frac{( ln ( \frac{C x ( t 2 )}{Cx ( t 1 )} ) * ( C s ( t 1 ) - C s ( t 2 ) )}{( C x ( t 2 ) - C x ( t 1 ) ) * ( t 2 - t 1 )}$$

i.e., qs=[ln(biomass concentration at t2/biomass concentration at t1)*(sugar concentration at t1−sugar concentration at t2)]/[(biomass concentration at t2−biomass concentration at t1)*(time t2−time t1)]

based on:

dCx/dt=mu*Cx

dCx/dt=qs*Ysx*Cx

qs=mu/Ysx

Mu=ln(Cx(t2)/Cx(t1))/(t2−t1)

Ysx=(Cx(t2)−Cx(t1)/(Cs(t1)−Cs(t2)

Biomass specific productivity (qp, gram product per gram cells per hour)

$$q p = \frac{( ln ( \frac{C x ( t 2 )}{Cx ( t 1 )} ) * ( C p ( t 2 ) - C p ( t 1 ) )}{( C x ( t 2 ) - C x ( t 1 ) ) * ( t 2 - t 1 )}$$

qp=[ln(biomass concentration at t2/biomass concentration at t1)*(product concentration at t2−product concentration at t1)]/[(biomass concentration at t2−biomass concentration at t1)*(time t2−time t1)]

based on:

qp=qs*Ysp

qp=[(mu/biomass yield)]*[(product concentration at t2−product concentration at t1)/(sugar concentration at t1−sugar concentration at t2)]

qp=(ln(biomass concentration at t2/biomass concentration at t1)/(time of t2−time of t1)/[(biomass concentration at t2−biomass concentration at t1)/(sugar concentration at t1−sugar concentration at t2)])*[(product concentration at t2−product concentration at t1)/(sugar concentration at t1−sugar concentration at t2)]

qp=ln(Cxt2/Cxt1)/(t2−t1)/Cxt2−Cxt1/Cst2−Cst1*Cpt2−Cpt1/Cst1−Cst2

Removing Cs's and simplifying to:

qp=ln(Cxt2/Cxt1)/(t2−t1)/((Cxt2−Cxt1)*(Cpt2−Cpt1))

The following parameters Rs and Rp are process rate parameters, distinguished from the above microbe rate parameters (qs and qp). One difference is that a microbe rate parameter is a per-cell metric, whereas a process parameter is a collective rate parameter dependent upon the number of cells (e.g., Rs=qsCx).

Volumetric sugar conversion (Rs, mmol sugar per liter per hour)

$$R s = \frac{( C s ( t 1 ) - Cs ( t 2 ) )}{( t 2 - t 1 )}$$

Rs=(sugar concentration at t1−sugar concentration at t2)/(time at t2−time at t1)

Volumetric productivity (Rp, mmol product per liter per hour)

$$R p = \frac{( C p ( t 2 ) - C p ( t 1 ) )}{( t 2 - t 1 )}$$

Rp=(product concentration at t2−product concentration at t1)/(time at t2 time at t1)

### Prophetic Example

The following is a prophetic example that accounts for the exponential growth behavior of microbes.

Glucose consumption, biomass formation and product formation were modeled for microbes with a variety of sugar uptake rates, biomass yields and product yields, using the following kinetic growth model formulas:

Biomass-specific sugar uptake rate (qs), dependent on sugar concentration:

qs=qs,max*Cs/(Ks+Cs)

Sugar consumption (dCs) per time interval (dt), dependent on biomass specific sugar uptake rate and biomass concentration, and sugar feed rate:

dCs/dt=−qs*Cx+Fs

Biomass production (dCx) per time interval (dt), dependent on biomass specific sugar uptake rate, sugar dissimilation for maintenance, biomass concentration, and biomass yield:

dCx/dt=qs*Cx*Ysx,max

Product formation (dCx) per time interval (dt), dependent on biomass specific sugar uptake rate, sugar dissimilation for maintenance, biomass concentration, and product yield:

dCx/dt=qs*Cx*Ysp

Some parameters are assigned as follows:

| Parameter | Default value | Unit | Description |
|---|---|---|---|
| Cₓ(0) | 1 | gX/L | Starting biomass concentration |
| Cₛ(0) | 30 | gS/L | Starting sugar concentration |
| Fs | 0.5 | gS/L/h | Sugar feed rate |
| q_{s, max} | 0.4-0.7 | gS/gX/h | Maximum sugar uptake rate |
| Kₛ | 0.5 | gS/L | Affinity value for sugar uptake |
| | | | rate |
| Y_{sx, max} | 0.05-0.15 | gX/gS | Maximum biomass yield |
| Yₛₚ | 0.525-0.675 | gP/gS | Product yield |

Input parameters for the model are variable sugar uptake rate, variable biomass yield (Ysx), variable product yield (Ysp), and some constant parameters.

Table A below shows the variable (maximum) sugar uptake rate (qs) used in hypothetical scenarios A-G:

| Scenario | Sugar uptake rate qs (g sugar/g cells/h) |
|---|---|
| A | 0.4 |
| B | 0.45 |
| C | 0.5 |
| D | 0.55 |
| E | 0.6 |
| F | 0.65 |
| G | 0.7 |

Table B below shows variable biomass yield (Ysx) and variable product yield (Ysp) (trade-off values) used in hypothetical scenarios 1-9.

| Scenario | Biomass yield Ysx (gX/gS) | Product yield Ysp (gP/gS) |
|---|---|---|
| 1 | 0.049286018 | 0.675 |
| 2 | 0.061607522 | 0.65625 |
| 3 | 0.073929026 | 0.6375 |
| 4 | 0.086250531 | 0.61875 |
| 5 | 0.098572035 | 0.6 |
| 6 | 0.11089354 | 0.58125 |
| 7 | 0.123215044 | 0.5625 |
| 8 | 0.135536548 | 0.54375 |
| 9 | 0.147858053 | 0.525 |

Table C below shows constant parameters used for the example:

| parameter | Value | Units |
|---|---|---|
| Initial cell concentration Cx0 | 1 | G cells/L |
| Initial sugar concentration | 30 | G sugar/L |
| Cs0 | | |
| Sugar feed rate | 0.5 | G Sugar/L/h |
| Sugar uptake affinity | 0.5 | G sugar/L |
| constant | | |

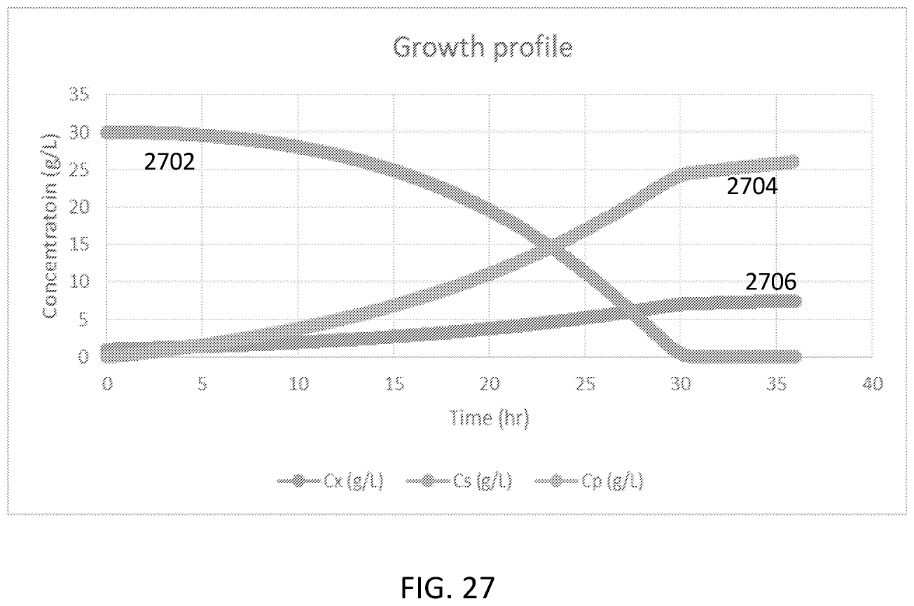

FIG. 27 plots sugar (Cs) 2702, product (Cp) 2704 and biomass (Cx) 2706 concentrations that were estimated over time using the kinetic growth model. See Table D for an example with a sugar uptake rate of 0.5 g sugar/g cells/h, a biomass yield of 0.1355 g biomass/g sugar, and a product yield of 0.544 g product/g sugar.

As show in Table D below, samples were simulated (including a low level of noise, 0.3%) using the kinetic growth model at different time points for a combination of the different scenarios A-G and 1-9. See below for modeled sugar, product and biomass concentrations after 20 hours of cultivation. The values were compared against the product yield (Ysp-ferm) of the strains in fermentations, which are assumed to be the same as the product yield (Ysp) of the microbe.

TABLE D

| | | | Plate Cs | Plate Cp | Plate Cx | Actual product |
|---|---|---|---|---|---|---|
| | | | after 20 h | after 20 h | after 20 h | yield Ysp in |
| Microbe | Microbe | Microbe | (g/L), with | (g/L), with | (g/L), with | fermenter (gP/gS), |
| qs (g/g/h) | Ysx (gX/gS) | Ysp (gP/gS) | noise | noise | noise | with noise |
| 0.4 | 0.049286018 | 0.675 | 30.540 | 6.489 | 1.469 | 0.678515 |
| 0.4 | 0.061607522 | 0.65625 | 29.923 | 6.670 | 1.622 | 0.663999 |
| 0.4 | 0.073929026 | 0.6375 | 29.314 | 6.800 | 1.792 | 0.637475 |
| 0.4 | 0.086250531 | 0.61875 | 28.902 | 6.938 | 1.971 | 0.616472 |
| 0.4 | 0.098572035 | 0.6 | 28.049 | 7.124 | 2.173 | 0.598028 |
| 0.4 | 0.11089354 | 0.58125 | 27.457 | 7.255 | 2.384 | 0.569804 |
| 0.4 | 0.123215044 | 0.5625 | 26.762 | 7.491 | 2.631 | 0.574604 |
| 0.4 | 0.135536548 | 0.54375 | 25.980 | 7.612 | 2.898 | 0.536564 |
| 0.4 | 0.147858053 | 0.525 | 25.150 | 7.782 | 3.194 | 0.525984 |
| 0.45 | 0.049286018 | 0.675 | 29.121 | 7.481 | 1.539 | 0.667671 |
| 0.45 | 0.061607522 | 0.6565 | 28.401 | 7.715 | 1.711 | 0.654201 |
| 0.45 | 0.073929026 | 0.638 | 27.541 | 7.987 | 1.925 | 0.642866 |
| 0.45 | 0.086250531 | 0.619 | 26.671 | 8.185 | 2.144 | 0.613148 |
| 0.45 | 0.098572035 | 0.6 | 25.874 | 8.462 | 2.390 | 0.605946 |
| 0.45 | 0.11089354 | 0.5815 | 24.933 | 8.693 | 2.659 | 0.587953 |
| 0.45 | 0.123215044 | 0.563 | 24.067 | 9.022 | 2.976 | 0.567682 |
| 0.45 | 0.135536548 | 0.544 | 23.041 | 9.269 | 3.323 | 0.541574 |
| 0.45 | 0.147858053 | 0.525 | 21.858 | 9.563 | 3.689 | 0.530735 |
| 0.5 | 0.049286018 | 0.675 | 27.400 | 8.536 | 1.620 | 0.665161 |
| 0.5 | 0.061607522 | 0.6565 | 26.426 | 8.816 | 1.825 | 0.644647 |
| 0.5 | 0.073929026 | 0.638 | 25.504 | 9.212 | 2.069 | 0.634518 |
| 0.5 | 0.086250531 | 0.619 | 24.611 | 9.538 | 2.322 | 0.618178 |
| 0.5 | 0.098572035 | 0.6 | 23.492 | 9.838 | 2.630 | 0.594583 |
| 0.5 | 0.11089354 | 0.5815 | 22.293 | 10.328 | 2.963 | 0.586114 |
| 0.5 | 0.123215044 | 0.563 | 20.841 | 10.726 | 3.351 | 0.56512 |
| 0.5 | 0.135536548 | 0.544 | 19.592 | 11.146 | 3.774 | 0.540532 |
| 0.5 | 0.147858053 | 0.525 | 18.085 | 11.543 | 4.250 | 0.526556 |
| 0.55 | 0.049286018 | 0.675 | 25.811 | 9.628 | 1.689 | 0.660924 |
| 0.55 | 0.061607522 | 0.6565 | 24.845 | 10.053 | 1.943 | 0.647998 |
| 0.55 | 0.073929026 | 0.638 | 23.641 | 10.513 | 2.216 | 0.638271 |
| 0.55 | 0.086250531 | 0.619 | 22.276 | 11.038 | 2.543 | 0.6244 |
| 0.55 | 0.098572035 | 0.6 | 20.805 | 11.544 | 2.901 | 0.602668 |
| 0.55 | 0.11089354 | 0.5815 | 19.268 | 12.030 | 3.301 | 0.5724 |
| 0.55 | 0.123215044 | 0.563 | 17.623 | 12.634 | 3.756 | 0.548298 |
| 0.55 | 0.135536548 | 0.544 | 15.779 | 13.209 | 4.275 | 0.549351 |
| 0.55 | 0.147858053 | 0.525 | 13.633 | 13.797 | 4.883 | 0.525766 |
| 0.6 | 0.049286018 | 0.675 | 23.957 | 10.765 | 1.783 | 0.673651 |
| 0.6 | 0.061607522 | 0.6565 | 22.841 | 11.396 | 2.059 | 0.658113 |
| 0.6 | 0.073929026 | 0.638 | 21.211 | 11.969 | 2.388 | 0.634771 |
| 0.6 | 0.086250531 | 0.619 | 19.636 | 12.575 | 2.779 | 0.625067 |
| 0.6 | 0.098572035 | 0.6 | 17.886 | 13.249 | 3.189 | 0.591891 |
| 0.6 | 0.11089354 | 0.5815 | 15.870 | 13.935 | 3.680 | 0.586068 |
| 0.6 | 0.123215044 | 0.563 | 13.837 | 14.767 | 4.250 | 0.562263 |
| 0.6 | 0.135536548 | 0.544 | 11.352 | 15.560 | 4.862 | 0.547687 |
| 0.6 | 0.147858053 | 0.525 | 8.725 | 16.352 | 5.639 | 0.520187 |
| 0.65 | 0.049286018 | 0.675 | 22.360 | 11.910 | 1.884 | 0.676242 |
| 0.65 | 0.061607522 | 0.6565 | 20.668 | 12.653 | 2.196 | 0.641914 |
| 0.65 | 0.073929026 | 0.638 | 18.839 | 13.411 | 2.557 | 0.645884 |
| 0.65 | 0.086250531 | 0.619 | 17.013 | 14.407 | 2.988 | 0.623918 |
| 0.65 | 0.098572035 | 0.6 | 14.603 | 15.227 | 3.506 | 0.598114 |
| 0.65 | 0.11089354 | 0.5815 | 12.223 | 16.191 | 4.059 | 0.578762 |
| 0.65 | 0.123215044 | 0.563 | 9.515 | 17.198 | 4.766 | 0.552749 |
| 0.65 | 0.135536548 | 0.544 | 6.504 | 18.231 | 5.515 | 0.54228 |
| 0.65 | 0.147858053 | 0.525 | 3.319 | 19.183 | 6.442 | 0.522942 |
| 0.7 | 0.049286018 | 0.675 | 20.395 | 13.194 | 1.972 | 0.667681 |
| 0.7 | 0.061607522 | 0.6565 | 18.612 | 14.076 | 2.324 | 0.657479 |
| 0.7 | 0.073929026 | 0.638 | 16.273 | 15.152 | 2.737 | 0.640358 |
| 0.7 | 0.086250531 | 0.619 | 13.845 | 16.164 | 3.242 | 0.616917 |
| 0.7 | 0.098572035 | 0.6 | 11.251 | 17.218 | 3.832 | 0.599234 |
| 0.7 | 0.11089354 | 0.5815 | 8.175 | 18.473 | 4.544 | 0.574191 |
| 0.7 | 0.123215044 | 0.563 | 4.897 | 19.759 | 5.335 | 0.562234 |
| 0.7 | 0.135536548 | 0.544 | 1.492 | 20.931 | 6.221 | 0.542419 |
| 0.7 | 0.147858053 | 0.525 | 0.058 | 20.941 | 6.870 | 0.517798 |

Next, correlations were calculated between:

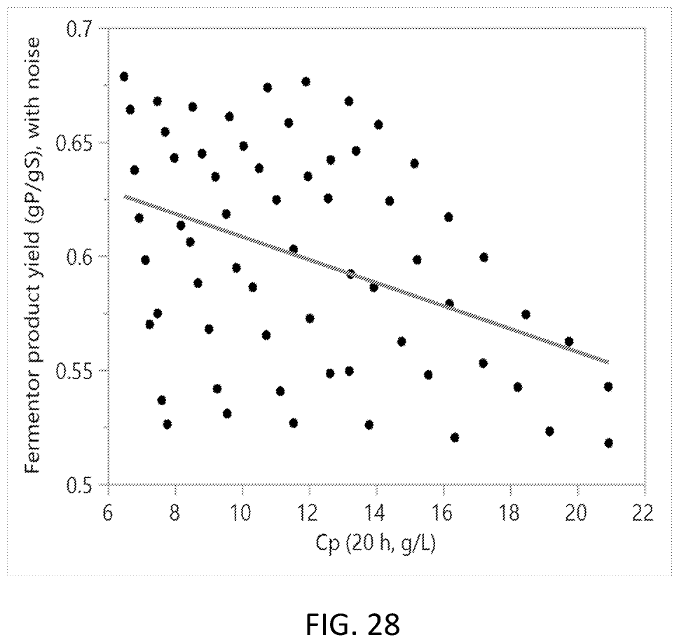

Fermenter yield (key performance indicator (“KPI”) of interest) and Cp after 20 hours in plates (poor correlation), as shown in FIG. 28, resulting in:

| | |
|---|---|
| RSquare | 0.16096 |
| RSquare Adj | 0.147205 |
| Root Mean Square Error | 0.044687 |

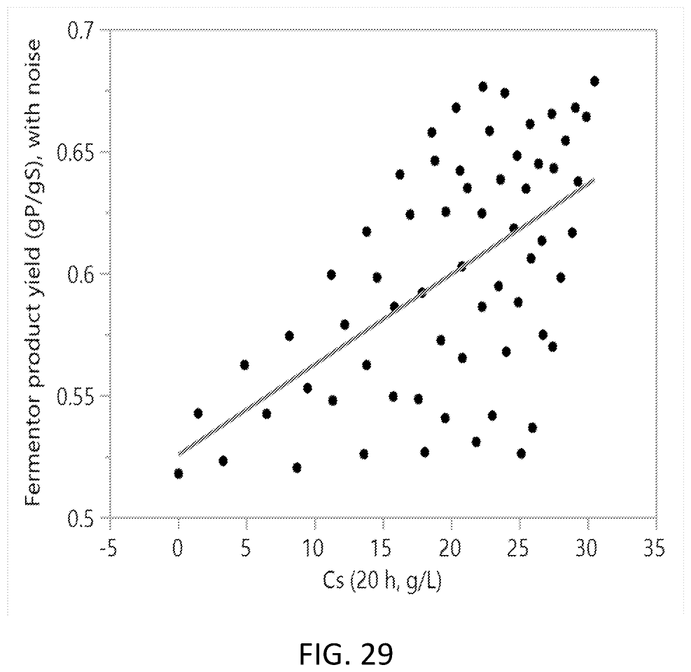

Fermenter yield (KPI of interest) and Cs after 20 hours in plates (poor correlation), as shown in FIG. 29, resulting in:

| | |
|---|---|
| RSquare | 0.325469 |
| RSquare Adj | 0.314411 |
| Root Mean Square Error | 0.040068 |

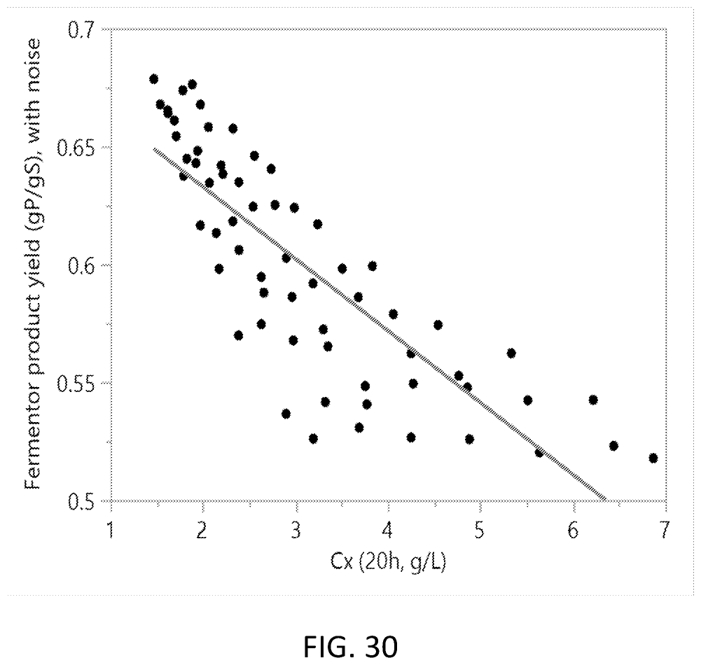

Fermenter yield (KPI of interest) and Cx after 20 hours in plates (poor correlation), as shown in FIG. 30, resulting in:

| | |
|---|---|
| RSquare | 0.678133 |
| RSquare Adj | 0.672857 |
| Root Mean Square Error | 0.027678 |

As shown above, when dealing with a variety of strains with different sugar uptake rates, biomass yields and product yields, and taking a mid-cultivation measurement, individual measurements of sugar, product and biomass do not correlate well with fermenter yield according to this prophetic example.

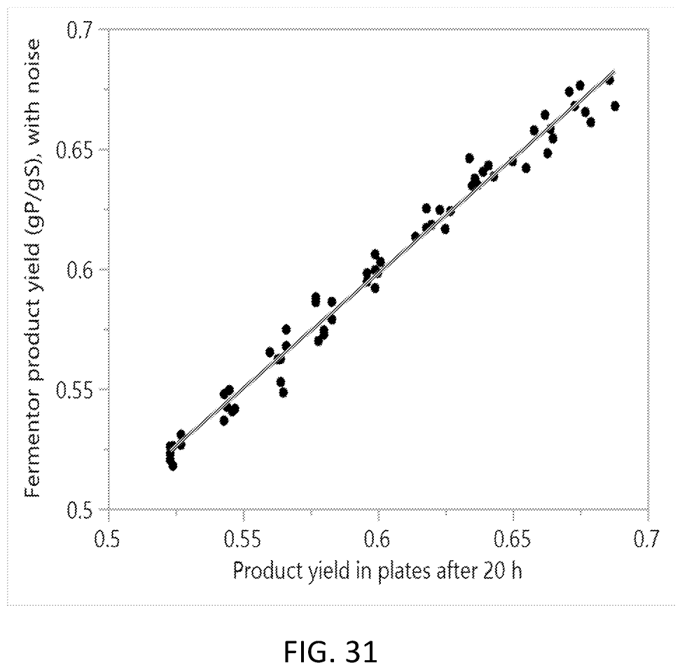

Statistics were also computed for fermenter (e.g., tank) yield (KPI of interest) and calculation of product yield in plates after 20 hours based on a function (e.g., quotient) of both Cp and Cs after 20 hours in plates, as shown in FIG. 31, resulting in a good correlation:

Ysp=Cp/(Total sugar fed in first 20 h−Cs)

| | |
|---|---|
| RSquare | 0.982442 |
| RSquare Adj | 0.982154 |
| Root Mean Square Error | 0.006464 |

As shown above, estimating product yield by the quotient of (product formed divided by sugar consumed), results in a much better correlation with fermenter yield. This ratio of microbe measurements is an estimate of a microbe property. Other examples of microbe properties are: sugar consumption rate, biomass yield, product yield (Ysp), growth rate, and cell-specific product formation rate.

As noted above, the prediction function may be represented as a weighted sum of variables:

PBP=a+b*PM1+c*PM2 . . . n*PMn

in which:
PBP=predicted bioreactor performance (e.g., y in other examples herein),
PMi=the ith plate data variable (e.g., first scale performance data variable xᵢ in other examples herein), which can be a measurement, or a function of measurements such as a combination of measurements or a statistical function of measurements (e.g., a statistical plate model), and
a, b, c, n, may be represented as mᵢ as in other examples herein

The results of the prophetic example immediately above show that, instead of using measurements such as Cp and Cs directly as the plate data variable PMi, the prediction engine can substitute for PMi one or more microbe properties derived from microbe measurements, such as a quotient or other combination of measurements, according to embodiments of the disclosure.

Transfer Function Development Tool

The transfer function development tool provides a reproducible, robust method for building the transfer function for a given experiment and for recording which strains are removed from the model. Having a development tool for the transfer function relies on the optimization of having a statistical model for predicting performance of lower-throughput performance from higher-throughput performance, and is an optimization in and of itself. Such a product wraps all the optimizations into one package that makes it straightforward for scientists to make use of the transfer function and all its optimizations.

According to embodiments of the disclosure, the raw plate-tank correlation transfer function is reduced to practice in a transfer function development tool (detailed below), along with optimizations such as outlier removal and inclusion of genetic factors. In embodiments of the disclosure, the transfer function development tool may incorporate further optimizations, include other statistical models, modifications to transfer function output, and considerations concerning the plate model.

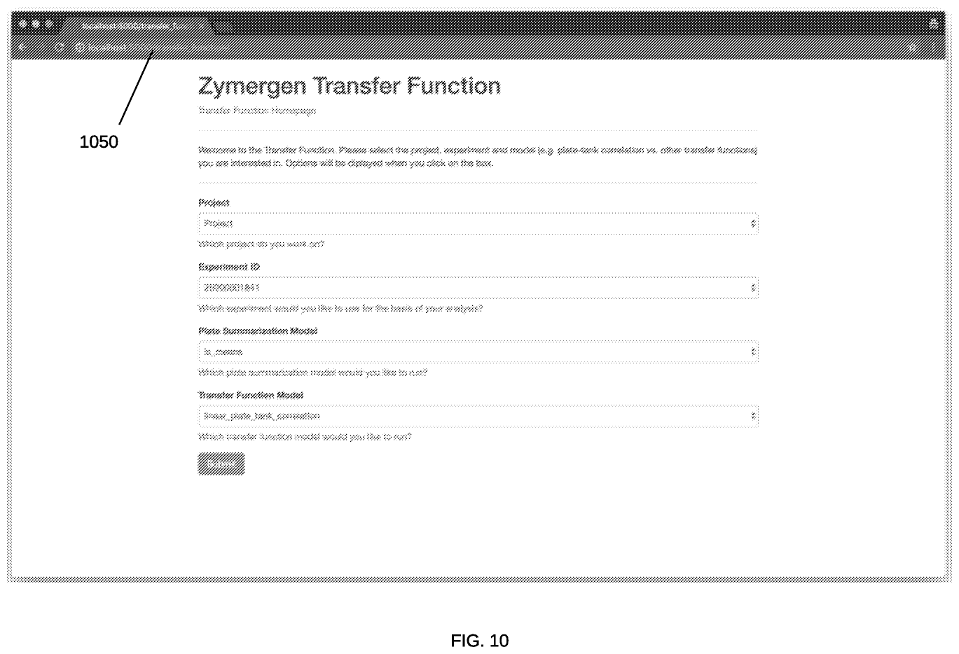

The transfer function development tool, in embodiments of the disclosure, takes high-throughput, smaller-scale performance data for a particular program, experiment, and measurement of interest, learns the appropriate model, and produces predictions for the next scale of work. FIGS. 10-15 show a series of screenshots for an embodiment of the user interface of the tool.

FIG. 10 illustrates a user interface having boxes for user entry of the project name, experiment ID, the selected plate summarization model (here, an LS means model), and the transfer function model to be used (here, a linear regression plate-tank correlation model).

Note the URL line in the address bar 1050 of the graphical user interface. This allows users to follow their progress through the process and confirm they have the correct information for the transfer function they want to implement. This setup is on the front end in the data models, and in the workflow infrastructure.

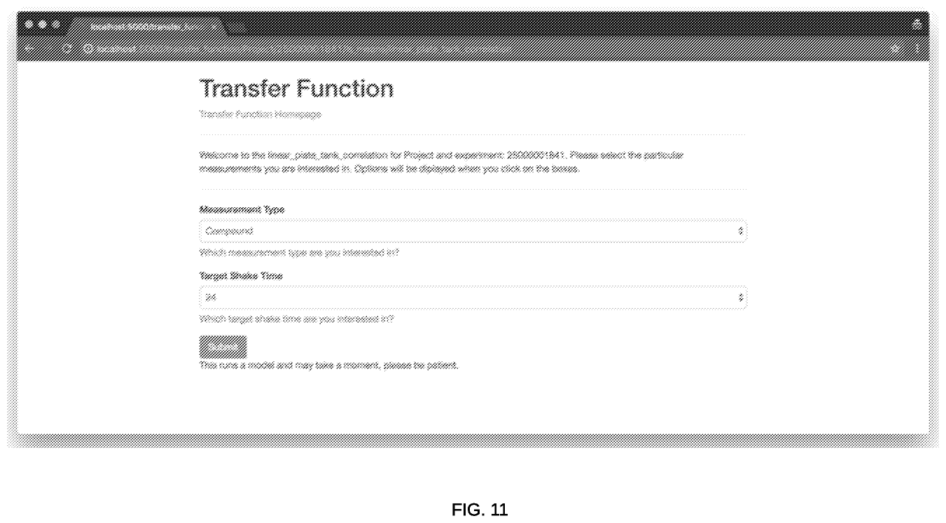

As illustrated in FIG. 11, after users enter their project, experiment, and model selections, they may choose the measurements they are interested in, e.g., amino acid yield (represented by “Compound”) in this example.

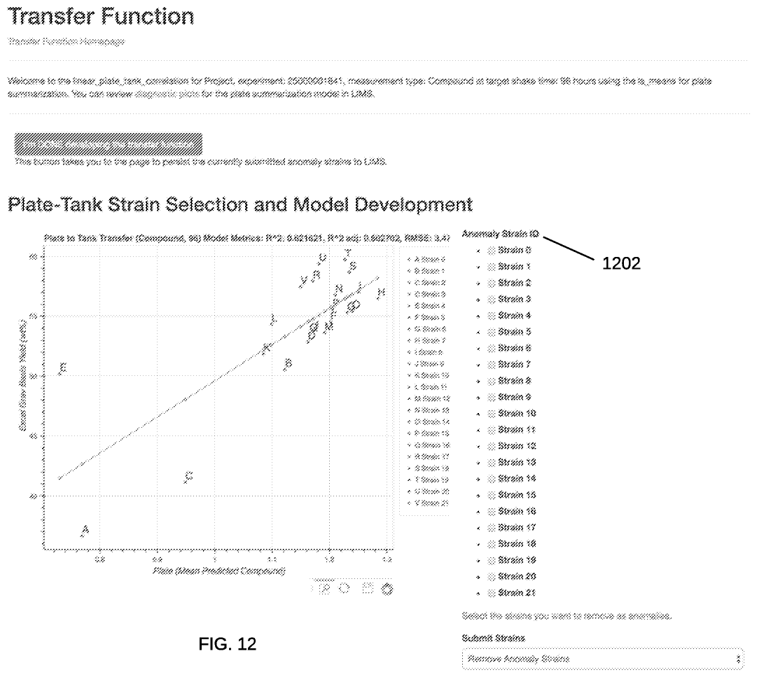

FIG. 12 illustrates a user interface for a plate-tank correlation transfer function after it has been developed for predicting amino acid performance at tank scale, according to embodiments of the disclosure. In this example, the transfer function is a linear fit line. The tool in this figure facilitates outlier evaluation. The user interface provides a list of strains 1202 (“Anomaly Strain ID”), identified by strain ID, along with checkboxes to enable a user to select strains for removal from the transfer function model.

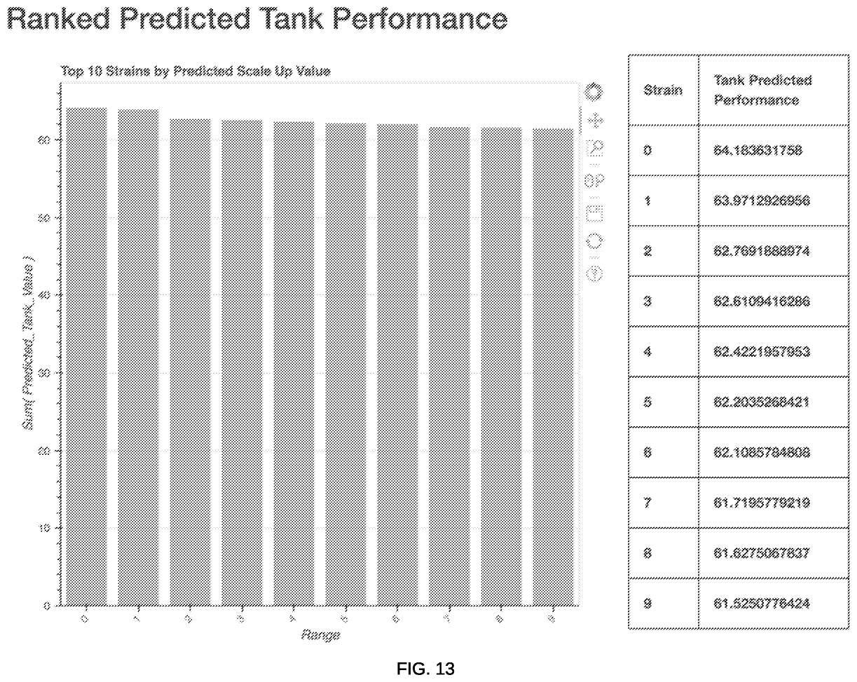

In FIG. 13, the user interface presents ten strains having the highest predicted performance based upon the transfer function with the outliers selected by the user having been removed from the model. Embodiments of the disclosure comprise selecting for manufacture and manufacturing strains in a gene manufacturing system based upon their predicted performance. Such a gene manufacturing system is described in International Application No. PCT/US2017/029725, International Publication No. WO2017189784, filed on Apr. 26, 2017, which claims the benefit of priority to U.S. nonprovisional application Ser. No. 15/140,296, filed on Apr. 27, 2016, both of which are hereby incorporated by reference in their entirety.

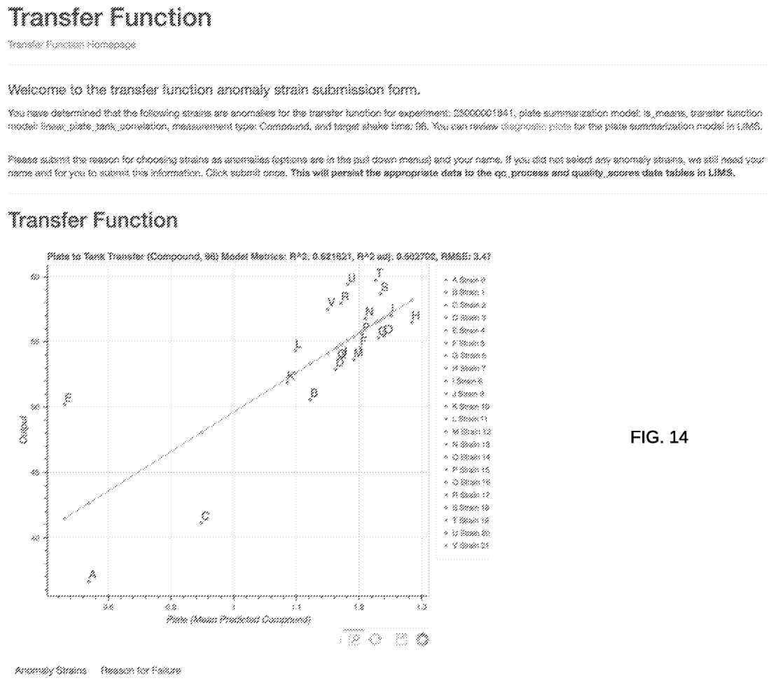

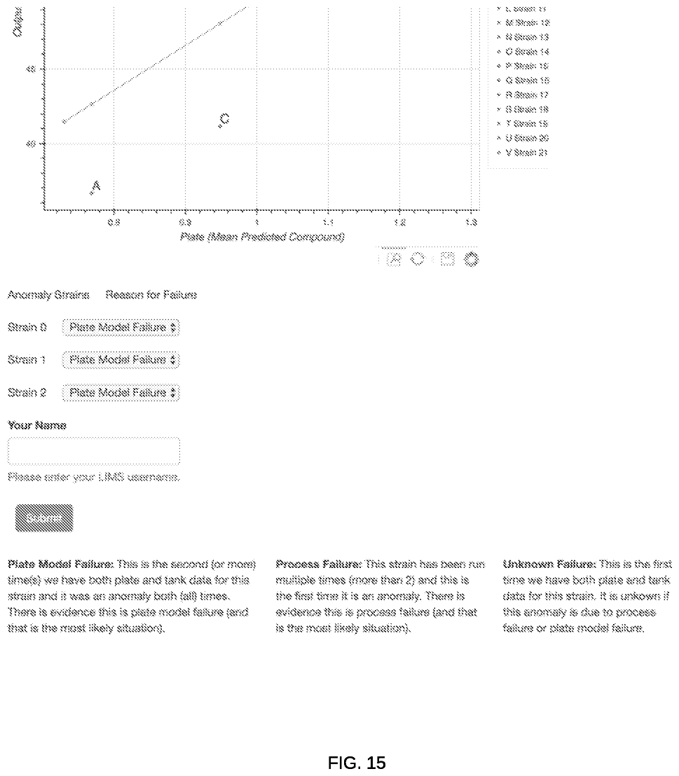

Referring to FIG. 14, the transfer function development tool returns a graphical representation of the chosen transfer function after user-selected outliers have been removed from the model, and (referring to FIG. 15) provides a mechanism to submit quality scores for the removed strains to a database, thus making the final results reproducible and providing a mechanism for users to track strains that are not working well with the existing plate model.

Machine Learning

Embodiments of the disclosure may apply machine learning (“ML”) techniques to learn the relationship between microbe performance at different scales, taking into consideration features such as genetic factors. In this framework, embodiments may use standard ML models, e.g. decision trees, to determine feature importance. Some features may be correlated or redundant, which can lead to ambiguous model fitting and feature inspection. To address this issue, dimensional reduction may be performed on input features via principal component analysis. Alternatively, feature trimming may be performed.

In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes, exhibited by training data. The result of the learning is then used to predict whether new data will exhibit the same patterns, categories, statistical relationships or other attributes.

Embodiments of the disclosure may employ other supervised machine learning techniques when training data is available. In the absence of training data, embodiments may employ unsupervised machine learning. Alternatively, embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

Computing Environment

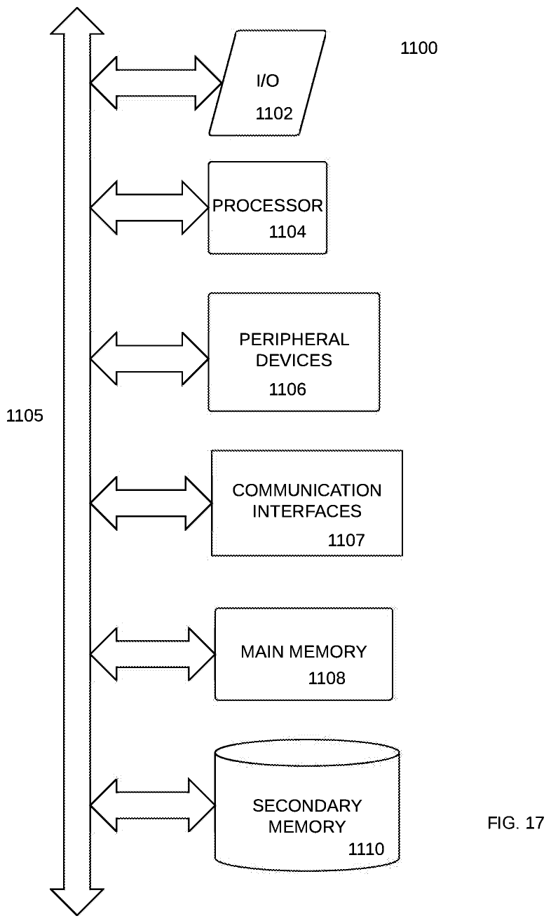

FIG. 16 illustrates a cloud computing environment according to embodiments of the present disclosure. In embodiments of the disclosure, the prediction engine software 1010 may be implemented in a cloud computing system 1002, to enable multiple users to generate and apply the transfer function according to embodiments of the present disclosure. Client computers 1006, such as those illustrated in FIG. 17, access the system via a network 1008, such as the Internet. The system may employ one or more computing systems using one or more processors, of the type illustrated in FIG. 17. The cloud computing system itself includes a network interface 1012 to interface the software 1010 to the client computers 1006 via the network 1008. The network interface 1012 may include an application programming interface (API) to enable client applications at the client computers 1006 to access the system software 1010. In particular, through the API, client computers 1006 may access the prediction engine.

A software as a service (SaaS) software module 1014 offers the system software 1010 as a service to the client computers 1006. A cloud management module 10110 manages access to the system 1010 by the client computers 1006. The cloud management module 1016 may enable a cloud architecture that employs multitenant applications, virtualization or other architectures known in the art to serve multiple users.

FIG. 17 illustrates an example of a computer system 1100 that may be used to execute program code stored in a non-transitory computer readable medium (e.g., memory) in accordance with embodiments of the disclosure. The computer system includes an input/output subsystem 1102, which may be used to interface with human users and/or other computer systems depending upon the application. The I/O subsystem 1102 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the prediction engine may be implemented with a computer system like that of computer system 1100.

Program code may be stored in non-transitory media such as persistent storage in secondary memory 1110 or main memory 1108 or both. Main memory 1108 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 1104 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 1104. The processor(s) 1104 may include graphics processing units (GPUs) for handling computationally intensive tasks.

The processor(s) 1104 may communicate with external networks via one or more communications interfaces 1107, such as a network interface card, WiFi transceiver, etc. A bus 1105 communicatively couples the I/O subsystem 1102, the processor(s) 1104, peripheral devices 1106, communications interfaces 1107, memory 1108, and persistent storage 1110. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 1100. In particular, the elements of the prediction engine and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 16.

Those skilled in the art will recognize that, in some embodiments, some of the operations described herein may be performed by human implementation, or through a combination of automated and manual means. When an operation is not fully automated, appropriate components of the prediction engine may, for example, receive the results of human performance of the operations rather than generate results through its own operational capabilities.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they are disclose essential matter.

Although the disclosure may not expressly disclose that some embodiments or features described herein may be combined with other embodiments or features described herein, this disclosure should be read to describe any such combinations that would be practicable by one of ordinary skill in the art. The user of “or” in this disclosure should be understood to mean non-exclusive or, i.e., “and/or,” unless otherwise indicated herein.

In the claims below, a claim n reciting “any one of the preceding claims starting with claim x,” shall refer to any one of the claims starting with claim x and ending with the immediately preceding claim (claim n−1). For example, claim 35 reciting “The system of any one of the preceding claims starting with claim 28” refers to the system of any one of claims 28-34.

1-137. (canceled)

138. One or more non-transitory computer-readable media storing instructions for improving performance of an organism with respect to a phenotype of interest at a second scale based upon measurements at a first scale, wherein the instructions, when executed by one or more computing devices, cause at least one of the one or more computing devices to:
a. access first scale performance data that is based at least in part upon observed first performance of one or more first organisms at a first scale and second scale performance data that is based at least in part upon observed second performance of one or more second organisms at a second scale larger than the first scale, wherein the first scale performance data is based at least in part upon a first scale statistical model; and
b. generate a prediction function based at least in part upon the relationship of the second scale performance data to the first scale performance data, wherein the prediction function is applicable to performance data observed for one or more test organisms with respect to the phenotype of interest at the first scale to generate second scale predicted performance data for the one or more test organisms at the second scale.

139. The one or more non-transitory computer-readable media of claim 138, wherein the prediction function is based at least in part upon a weighted sum of one or more first scale performance variables, and at least one of the first scale performance variables is based on a combination of two or more measurements of organism performance.

140. The one or more non-transitory computer-readable media of claim 138, wherein the first scale statistical model represents organism features at the first scale.

141. The one or more non-transitory computer-readable media of claim 138, wherein the organism features comprise process conditions, media conditions, or genetic factors.

142. The one or more non-transitory computer-readable media of claim 138, wherein at least one organism feature relates to organism location.

143. The one or more non-transitory computer-readable media of claim 138, wherein generating the prediction function further comprises incorporating one or more factors to reduce error of the prediction function.

144. The one or more non-transitory computer-readable media of claim 138, wherein generating the prediction function further comprises adjusting for at least one genetic factor.

145. The one or more non-transitory computer-readable media of claim 138, storing further instructions for:
a. modifying the prediction function by one or more factors from a set of factors; and
b. excluding, from consideration in generating the prediction function, a first candidate outlier organism which, if included in generating the prediction function, would result in the modified prediction function having a leverage metric that fails to satisfy a leverage condition.

146. The one or more non-transitory computer-readable media of claim 138, storing further instructions for:
a. modifying the prediction function by one or more factors from a set of factors; and
b. if a leverage metric for the modified prediction function with respect to a first candidate outlier organism satisfies a leverage condition, using the modified prediction function as the prediction function.

147. The one or more non-transitory computer-readable media of claim 138, wherein a first candidate outlier organism is represented in the first scale performance data and the second scale performance data, the one or more test organisms comprise the first candidate outlier organism, and the second scale predicted performance data represents predicted performance of the first candidate outlier organism at the second scale.

148. The one or more non-transitory computer-readable media of claim 138, wherein modifying the prediction function comprises incorporating or removing the one or more factors respectively into or from the prediction function.

149. The one or more non-transitory computer-readable media of claim 138, wherein the one or more factors comprise a genetic factor.

150. The one or more non-transitory computer-readable media of claim 138, wherein generating the prediction function comprises training a machine learning model using the first scale performance data and the second scale performance data.

151. The one or more non-transitory computer-readable media of claim 138, wherein the first scale performance data for the one or more first organisms represents the output of a first scale statistical model, the one or more non-transitory computer-readable media storing further instructions for:
a. comparing predicted performance for the one or more first organisms at the second scale with the second scale performance data; and
b. adjusting parameters of the first scale statistical model based at least in part upon the comparison.

152. The one or more non-transitory computer-readable media of claim 138, wherein the first scale is a plate scale and the second scale is a tank scale.

153. The one or more non-transitory computer-readable media of claim 138, wherein the one or more second organisms are a subset of the one or more first organisms.

154. The one or more non-transitory computer-readable media of claim 138, storing further instructions for applying the prediction function to performance data observed for the one or more test organisms with respect to a phenotype of interest at the first scale to generate the second scale predicted performance data for the one or more test organisms at the second scale.

155. The one or more non-transitory computer-readable media of claim 138, storing further instructions for manufacturing at least one of the one or more test organisms based at least in part upon the second scale predicted performance.

156. One or more non-transitory computer-readable media storing instructions for improving performance of an organism with respect to a phenotype of interest at a second scale based upon observed performance of organisms at a first scale smaller than the second scale, wherein the instructions, when executed by one or more computing devices, cause at least one of the one or more computing devices to:
a. access a prediction function, wherein the prediction function is based at least in part upon the relationship of second scale performance data to first scale performance data, the first scale performance data is based at least in part upon a first scale statistical model and observed first performance of one or more first organisms at a first scale, and the second scale performance data represents observed second performance of one or more second organisms at a second scale larger than the first scale; and
b. apply the prediction function to one or more test organisms at the first scale to generate second scale predicted performance data for the one or more test organisms at the second scale.

157. The one or more non-transitory computer-readable media of claim 156, wherein the prediction function is based at least in part upon a weighted sum of one or more first scale performance variables, and at least one of the first scale performance variables is based on a combination of two or more measurements of organism performance.

158. The one or more non-transitory computer-readable media of claim 156, wherein the prediction function incorporates one or more genetic factors to reduce error of the prediction function.

159. The one or more non-transitory computer-readable media of claim 156, wherein the prediction function excludes influence by a first candidate outlier organism which, if included in generating the prediction function, would result in a modified prediction function having a leverage metric that fails to satisfy a leverage condition, wherein the modified prediction function incorporates modificiation by one or more factors into the prediction function.

160. The one or more non-transitory computer-readable media of claim 156, wherein the prediction function is generated by training a machine learning model using the first scale performance data and the second scale performance data.

161. The one or more non-transitory computer-readable media of claim 156, wherein the first scale is a plate scale and the second scale is a tank scale.

162. The one or more non-transitory computer-readable media of claim 156, wherein the one or more second organisms are a subset of the one or more first organisms.

163. The one or more non-transitory computer-readable media of claim 156, storing further instructions for manufacturing at least one of the one or more test organisms based at least in part upon the second scale predicted performance.

164. One or more non-transitory computer-readable media storing instructions for improving performance of an organism with respect to a phenotype of interest at a second scale based upon observed performance at a first scale smaller than the second scale, wherein the instructions, when executed by one or more computing devices, cause at least one of the one or more computing devices to:
a. receiving first user input representing selection of a first scale statistical model that represents organism features at the first scale;
b. receiving second user input representing selection of a prediction function;
c. receiving third user input representing selection of a type of performance data for the phenotype of interest; and
d. providing, for graphic display, a prediction function, the prediction function for providing second scale predicted performance data of the selected type for one or more test organisms at the second scale, based upon application of the prediction function to performance data observed for one or more test organisms at the first scale.

165. The one or more non-transitory computer-readable media of claim 164, storing further instructions for providing, for graphic display, the second scale predicted performance data for one or more test organisms at the second scale.

166. The one or more non-transitory computer-readable media of claim 164, wherein the first scale performance data is generated using the first scale statistical model.

167. The one or more non-transitory computer-readable media of claim 164, storing further instructions for receiving user input representing user selection of one or more factors to be used in generating the prediction function.

168. The one or more non-transitory computer-readable media of claim 164, wherein the one or more factors include one or more genetic factors.

169. The one or more non-transitory computer-readable media of claim 164, storing further instructions for producing at least one of the one or more test organisms.
